(19) United States
(12) Patent Application Publication
(10) Pub. No.: US 2011/0257028 A1
(43) Pub. Date: Oct. 20, 2011

(54) Method for selectively binding a substrate to sorbents by way of at least bivalent bonds

(75) Inventor: Klaus Gottschall, Ludwigshafen (DE)

(73) Assignee: INSTRACTION GmbH, Ludwigshafen (DE)

(21) Appl. No.: 12/807,578

(22) Filed: Sep. 9, 2010

Related U.S. Application Data
(63) Continuation of application No. 10/550,756, filed on Jul. 24, 2006, now abandoned.

(30) Foreign Application Priority Data
Mar. 25, 2003 (DE) 10313324.0
Dec. 15, 2003 (DE) 10358696.2

(51) Int. Cl. C40B 30/04; C07H 1/00; C07K 1/107; C07K 16/00; C12N 7/00; B01D 15/08; G01N 33/53; G01N 33/573; C12Q 1/70; C40B 40/12; C40B 40/06; C40B 40/04; C12N 9/96; C12N 1/00

(52) U.S. Cl. 506/9; 435/188; 530/333; 536/22.1; 530/402; 530/395; 530/391.1; 536/124; 435/235.1; 435/243; 436/501; 435/7.8; 435/5; 435/7.1; 506/19; 506/16; 506/15; 210/198.2

(57) ABSTRACT

A method for the manufacture of at least one sorbent having at least two different groups, which are capable of binding, for the selective binding of a substrate, characterized in that it comprises the steps (i) to (ii):
(i) determining at least two groups capable of binding a sorbent from a synthetic or natural first substrate,
(ii) respectively applying at least two different groups capable of binding a second synthetic or natural substrate to one respective carrier, thereby forming at least one sorbent, whereby the groups are the same groups of step (i) or are groups that are complementary thereto, and the second substrate of step (ii) is the same or different from the first substrate according to step (i),
   and whereby the groups are determined such that the contributions of the Gibbs energies of the individual groups to the non-covalent bond with the second substrate yield a negative value of the Gibbs energy ΔG, such that a binding strengthening occurs that results in an improved separation selectivity with respect to at least one substance to be separated off.

The invention relates to a method for the manufacture of at least one sorbent for the selectively binding of a substrate with at least two different groups capable of binding as well as to a method for the selectively binding of said substrate by way of said sorbents. Said sorbent is determined from a collection of sorbents on the surfaces of which are each at least two different groups capable of binding that are gained by dissection of synthetic or natural substrates into components containing said groups. In particular, the method for the selectively binding is suitable for the isolation of synthetic or also natural agents as well as for the characterization and identification of the function and the properties of said agents. Another subject of the invention is also a sorbent/substrate complex which is obtained in the selectively binding of said substrate. Furthermore, the invention also relates to a combinatorial library comprising sorbents and substrates, preferably having each at least two different amino acid residues, sugar residues, nucleotide residues, nucleoside residues, pyrimidine residues and/or purine base residues as groups capable of binding. The method of the selectively binding as well as the combinatorial library can be used for the detection of substrate/receptor interactions, for the agent screening, for the selective separation of isomeric compounds, for the selective separation as well as for the purification of substrates.

It is already known from the bioaffinity chromatography to chemically immobilize substances on the surface of an insoluble carrier with high molecular weight that have a particularly high affinity to specific biomolecules. Then, they are capable of binding said biomolecules.

Mostly, the substances to be immobilized on the carrier are biopolymers. On the other hand, it is also possible to attach substances with low molecular weight to the surface with which the binding or retention of biopolymers is possible.

Thereby, in general, there is only a sufficiently high affinity, if sorbent and substrate have groups being complementary to each other and being capable of forming a bond. For example, complementary groups are hydrophilic groups which can interact with each other by way of hydrogen bonds or dipoles or polypoles, whereby the binding takes place.

It is already known that biological systems can simultaneously interact with each other by way of several molecular contact sites (M. Withesides et al., Angew. Chem. 1998, 110, 2908-2953).

Furthermore, from WO 00/32649 polymers are known as sorbents for the separation of substrates as well as methods for the separation of substrates by way of said sorbents. Here, the separation is made possible via at least two different types of interactions. The group of the sorbent capable of binding which acts as receptor can be a single type of groups, however, can also be two or more different types of groups.

Furthermore, the patent documents WO 00/32648, WO 01/38009 as well as WO 00/78825 disclose sorbent/substrate interactions providing good conditions for at least bivalently binding.

In these methods, for the targetedly binding of a substrate, also the suited biopolymer must be known and must be producible, if it is to be used as a part of the sorbent. If, conversely, biopolymers are bound on the sorbent by way of low-molecular substances, the latter ones must also be known and must be immobilizable on the carrier without changing the binding properties.

A method for providing synthetic groups on a polymeric compound for the binding of biologically or pharmacologically active substances is also known. For this, template molecules being biologically or pharmacologically active substances are fixed at the polymeric compound. After attachment of the reactive functional groups to the polymeric compound for the binding of substrates, the template molecules are re-detached (WO 00/13016).

Furthermore, a method for the selective separation of a selected organic compound is also known. For this, groups are applied on the surface of a carrier which are complementary to the groups of the compound to be separated off. Preferably, the compounds to be separated off are macromolecules having ionizable groups. The binding groups on the surface of the carrier are inversely charged to the groups of the macromolecules. However, there is only one type of groups on the sorbent by means of which the binding takes place (WO 93/19844).

Furthermore, also the US 2002/0155509 A1 discloses a method which finally can be used for the selective separation of a substrate from a substrate mixture. For this, the substrate mixture is brought into contact with different sorbents and eluents. By means of desorption spectrometry, it can be determined whether and how strong substrates are bound to the sorbents with the selected sorbent/eluent combinations. Sorbent and eluent can be varied as long as a suited sorbent/eluent combination is found which allows for the selective separation of a substrate (thereby, the terms “sorbent” and “substrate” are used in a manner which differs from the definition used in the US 2002/0155509 A1 in the definition on which the present patent application is based, and which is set forth below).

It is also already known to immobilize polar groups together with long-chain alkyl radicals on the surface of the carrier, whereby sorbents with at least two different groups capable of binding are produced. Here, in a first reaction step, chlorosilanes which are preferably substituted with medium-chain or long-chain alkyl radicals, such as a C₈- or C₁₈-radicals, are reacted with OH groups of the surface of the carrier, for example silicol groups of the silica gel, whereby said alkyl radicals are immobilized on the surface of the carrier. Then, in a second step, the surface of the carrier is reacted with trimethoxysilanes or triethoxysilanes followed by a hydrolysis step under separation of alcohol and formation of a silicol group. Furthermore, it is also possible to react silicon compounds such as alkyltrialkoxysilanes, such as octadecyltrimethoxysilane, with OH groups of the surface of the carrier, whereupon at first the alkyl radical is immobilized. Non-reacted alkoxy residues can then be hydrolyzed under formation of silicol groups, whereupon the second group capable of binding is generated. In particular, it is believed that said sorbents are usable for the binding of substrates from aqueous solutions (Column Watch, LC*GC Europe, December 2002, page 780-786).

If substrates with yet unknown structure and/or binding properties are to be separated off by way of sorbents, the methods which are described in the prior art, in general do not allow to targetedly predict whether and how good a certain sorbent is capable or not capable of the selectively binding of the substrate. Here, mostly in complex experiments, it has to be analyzed whether known sorbents are suitable or are not suitable to the selectively binding of said substrate. Then, the finding of a suitable substrate is rather a coincidence.

Consequently, it was the object of the invention to provide a method for the manufacture of a sorbent with which the targeted separation of a substrate, preferably a substrate with physiological activity, is possible from a substrate mixture. Furthermore, it was the object of the invention to provide a method which allows the targeted separation of said substrate from a substrate mixture by way of said sorbent.

These objects could be solved by way of at least one sorbent which contains at least two different groups capable of binding that can complementarily bivalently interact with at least two groups at the substrate. Through this, compared to the monovalent interaction which is few-selective to non-selective, a strengthening takes place. In consequence of, the target compound is strongerly retained for a multiple by the sorbent than only monovalently binding competitors, whereby, compared to said competitors, the selectively binding is achieved. Compared to other polyvalent competing substrates, the selectively binding can be achieved by an optimized sorbent, whose necessary properties can be determined by way of a collection of sorbents.

Thus, the object of the invention is a method for the manufacture of at least one sorbent having at least two different groups, which are capable of binding, for the selective binding of a substrate, characterized in that it comprises the steps (i) to (ii):
(i) determining at least two groups capable of binding a sorbent from a synthetic or natural first substrate,
(ii) respectively applying at least two different groups capable of binding a second synthetic or natural substrate to one respective carrier, thereby forming at least one sorbent, whereby the groups are the same groups of step (i) or are groups that are complementary thereto, and the second substrate of step (ii) is the same or different from the first substrate according to step (i).

Another object of the invention is also a method for the selectively binding of a substrate having at least two different groups, which are capable of binding, to at least one sorbent, characterized in that it comprises the steps (i) to (iv):
(i) determining at least two groups capable of binding a sorbent from a synthetis or natural first substrate,
(ii) respectively applying at least two different groups capable of binding a second synthetic or natural substrate to one respective carrier, thereby forming at least one sorbent, whereby the groups are the same groups of step (i) or are groups that are complementary thereto, and the second substrate of step (ii) is the same or different from the first substrate according to step (i),
(iii) contacting the at least one second substrate that is the same or different from the first substrate according to (i) with at least one sorbent of step (ii),
(iv) testing the binding strength of the at least one second substrate to the at least one sorbent of step (iii).

Thus, the invention allows to targetedly strengthen or also to targetedly weaken the bond between sorbents and substrates, whereby the selectivity of the binding of a sorbent to a substrate which is to be separated off from a substrate mixture can also be targetedly improved.

Consequently, the invention is based on a new separation principle for a substrate from a substrate mixture that fundamentally differs from the separation principles of the methods of the prior art, because it designs and realizes the promising separation selectivity for any substrate pair to be separated.

The separation principle of the present invention is based on the prediction, on the quantifiable estimation or on the measurement of the intensity of the non-covalent bond that is formed by way of interaction between at least two different groups capable of binding of the sorbent and substrate, respectively. The separation principles of the methods of the prior art are based on the fact that the separation is carried out by means of empirical methods which are roughly classified into the categories polar/nonpolar respectively hydrophilic/hydrophobic, and therefore is a random method. This is also confirmed by the separation success which, so far, frequently is not sufficient.

Preferably, the groups in step (ii) are the same groups as the groups of step (i) or are complementary to said groups.

In the meaning of the invention, the term substrate encompasses all substances of natural or synthetic origin that can be selectively bound. Preferably, these substances are agents, also compounds with physiological and/or biological activity in living vegetable or animal organisms. In principle, these substances are all natural and synthetically chemical and/or biological compounds having two or more groups capable of binding. Preferably, these are amino acids, oligopeptides, nucleotides, nucleosides, proteins, glycoproteins, antigens, antigen determinants, antibodies, carbohydrates, enzymes, co-enzymes, ferments, hormones, alkaloids, glycosides, steroids, vitamins, metabolites, viruses, microorganisms, substances of content of vegetable and animal tissue, cells, cell fragments, cell compartments, cell disruptions, lectins, flavylium compounds, flavones and isoflavones, as well as synthetic agents, like pharmaceuticals and plant protective agents.

In case of low-molecular agents to be bound, in the literature, said agents are frequently termed as ligands. Protein-like binding substances having a high molecular weight are frequently termed as receptor.

The term substrate encompasses also pre-stages which, as the case may be, can be suited as agent after further modification. Such potential agents are often termed as hits or leads, if, for their determination, they are derived from the used screening methods, or they are termed as scaffolds, needles or pharmacophores if they are derived from structure features.

Furthermore, said term substrate also encompasses resources, whose isolation, removal or winning from mixtures can be of economical benefit. Among said resources are also resources in low concentration and by-products, for example from process flows or waste flows. The resources can be organic, such as peptides, or metabolites from body liquids, or inorganic, such as radioactive metal ions or metal ions of the noble metals.

The term carrier encompasses materials that serve as carrier or scaffold for the groups to be bound. In applying said groups to the carrier, the sorbent is formed. For chromatographic applications, the sorbent is also termed as stationary phase.

The term sorbent encompasses any combination of carrier and at least two different groups capable of binding a substrate.

The term component means parts or fragments of substrates, preferably agents, each having at least one group capable of binding. Examples for such components are epitopes. The term component can also be identical to the term group capable of binding. In the following, the spatial arrangement of the components within a substrate is frequently denominated as binding site. For example, histidine is a component carrying as group capable of binding an imidazole residue that in turn contains amidine or imine groups as group capable of binding.

The term epitope denominates molecular regions of substrates. For example, the term epitope denominates a molecular region of an antigen that is capable of binding an antibody. Such binding sites of an antibody on an antigen are also denominated as antigen determinant.

The term (different) group capable of binding encompasses all groups capable of binding the sorbent and/or substrate by way of covalent or non-covalent interactions. In the English literature, said term is also termed binding site residue. By the way, these groups are all compounds or the residues of compounds which are described in the literature for being able to form non-covalent bonds. The term non-covalent bond is explained below.

Preferably, groups capable of binding are hydroxyl, carboxyl, amide, amino, i-butyl, phenyl, nitrophenyl, naphthyl, however also diol, hydroxyphenyl, carbonyl, imine, alkylene, alkinyl, indolyl and imidazolyl residues. Thus, a group capable of binding can contain at least one functional group. However, the groups capable of binding are not limited to functional groups.

A group capable of binding can also perform more than one form of an energetic interaction, that is it can undergo more than one type of non-covalent bond. For example, basically the indole residue is capable of simultaneously performing with suitable substances to be bound, ionic, van der Waals, 7′-n and disperse interactions. However, the indene residue lacks of ionic capability of interaction and the disperse interaction is weaklier developed.

Thereby, the individual contributions to the bond are also dependent on the solvent. They can be targetedly influenced by the choice of the solvent composition, the pH and the temperature. In general, the van der Waals interactions are less developed in organic solvents than in aqueous solvent mixtures. Compared to this, as a rule, the hydrogen bond interactions in aprotic solvents are strongly lowered with increasing water content.

The term different means that the groups have either a different elementary composition, or, that for the same elementary composition, the elements in the groups are differently linked, or the groups are differently chemically bonded. The difference concerning at least two groups capable of binding also includes the steric arrangement compared to a substance to be bound. Referring to this, for example, an arrangement concerns the differentiation of stereoisomers, in particular of diastereomers and enantiomers. For example, the hydroxyl groups in a cis arrangement are different to hydroxyl groups in a trans arrangement, or hydroxyl groups of a R form are different from those ones of a S form. Such differences can be detected by physical methods, for example by way of NMR spectroscopy, because such groups are magnetically non-equivalent and produce different resonance signals in the NMR spectrum. The detection can also be performed by means of X-ray structure analysis. Also, such groups are characterized in that they can have a different reactivity towards attacking reagents.

Thus, in particular, different groups capable of binding are such groups that each contribute to the interaction energy different contributions towards the substance to be bound (second substrate). Said interaction energy is also denominated as interaction Gibbs energy ΔG. By all means, such groups can be the same with respect to their constitution, configuration and conformation, however, can differ in their interaction contribution. For example, in glutamic acid derivatives, carboxyl groups can have a different interaction contribution. Also, rhamnose residues that are differently bonded may have a different interaction contribution which, for example, may be used for the separation of naringine and rutine.

In turn, different contributions, to the interaction Gibbs energy ΔG can have differently high enthalpy and entropy components, respectively. So, it is conceivable that two ionic interactions of the carboxyl groups that are contained in the substance to be bound, indeed contribute nearly the same contributions with respect to the enthalpy ΔH interaction, however, the second binding site has a relatively higher negative entropy contribution ΔS.

Conversely, it also happens that in a first and/or second substrate at least two groups capable of binding are directly adjoined being chemically the same or equivalent. The contributions thereof to the interaction, as the case may be, only gradually differ from each other, and are not longer distinguishable within the accuracy of measurement. The stoichiometric ratio of such groups among each other or in respect to further groups capable of binding, is taken into account in the manufacture of the sorbent by the degree of derivatization. For solutions or suspensions of the sorbent, said derivatization degree is also the measure for concentration specifications.

An example for an accumulation of same or energetically approximated equivalent groups capable of binding are the steroid receptors. For the binding contact to estradiol or progesterone, steroid receptors contain up to seven leucine residues, which non-polarly bind the ligand via their alkyl groups. Additionally, there are up to three polar binding sites consisting of arginine, glutamine (glutamic acid) and histidine. According to the invention, said natural receptors can simply be simulated by inserting i-pentyl radicals from methylvaleric acid, and polar groups, such as succinic acid amide as well as basic groups, such as amine or imidazole, in a suited concentration ratio.

Such sorbents are capable of strongly binding not only the target molecule estradiol in a suitable manner, but also a series of synthetic and natural substances which exhibit in physiological tests and in vivo estrogen-like activity. Among these substances are, for example, diethylstilbestrol and genistein.

Thereby, preferably, the sorbent as synthetically polymeric receptor is calibrated with such agents, but also with agents that are structurally related thereto which, however, are inactive, such as tamoxifene, testosterone, or catechine. The practical benefit is given if the substances that are well binding at the natural receptor also exhibit a strong binding at the sorbent, contrarily to substances already binding weakly or non-specifically at the model. In optimizing the structure, besides the ratio of the groups capable of binding, also the cross-linking degree is adjusted that regulates the extent and spatial condition of the binding sites.

Such sorbents bind from dissolved substance mixtures predominantly such substances or even exclusively such substances which also are strongly bound in the biological protein model. Thus, from substance mixtures of natural or synthetical origin, potential agents can be isolated in pure form in a fast and simple manner.

An important aspect of the invention is the largely free choice of the solvent in the method respectively use according to the invention. The ranking and the dimension of the differences in the bond energy between the strongly and weakly binding substances surprisingly remain largely unchanged, if one adds larger amounts of alcohol and additional acids or buffer to the aqueous eluent. Preferably, the addition of methanol considerably weakens the binding for all substances that are used in the calibration, without affecting the partition into the groups of strongly and weakly binding substances. The consequence is a considerably earlier elution under chromatography conditions. So, the substances of interest can be tested or isolated in a passable time, because by means of the addition of organic solvents, the bond constants are decreased by the power of ten compared to pure water or physiological buffer.

The term non-covalent bond means that the groups capable of binding can bind each other via ion pairs, hydrogen bonds, dipole-dipole interactions, charge transfer interactions, π-π interactions, cation-π-electron interactions, van der Waals interactions and disperse interactions, hydrophobic (lipophilic) interactions, complex formation, preferably complex formation of transition metal cations, as well as via combinations of said interactions.

The term complementary has the meaning that only such groups are capable of forming a bond that are suited to each other. Thereby, the interaction which causes the binding must be energetically favorable. The more developed the non-covalent bond of said groups is with each other, the stronger the substrate is bound to the at least one sorbent. Thereby, it is also possible that several groups can be complementary to one group. For example, the carboxyl group, the amine group and the amide group can be complementary to the hydroxyl group.

The term complementary groups also includes that such groups can be replaced by groups being structurally similar to the complementary groups or being structurally related to said groups. For example, it is possible to replace in a non-covalent bond that is based on π-π interaction, a naphthyl residue by an anthracene residue, whereby the contribution of the aromatic hydrocarbon to the binding strength of the non-covalent bond is further modified respectively increased. In an analogous matter, it is possible to increase the contribution of an indole residue in a disperse non-covalent bond by replacing by an acridine residue.

The strength of the interaction between complementary groups which, for example can be measured and expressed as bond constant, results from the contributions of the individual groups capable of binding. These individual contributions to the bond constant are not only dependent on the type of the non-covalent interaction, but also from the distances and the orientations (angle) of the groups interacting with each other as well as from the composition of the solvent. The individual types of interaction considerably differ of each other in energy, whereby the bond and therewith the Gibbs energy differently decrease with the distance between said groups.

Groups being complementary towards each other are also characterized in that the contributions of the Gibbs energies of the individual groups for the non-covalent bond result in an change of the Gibbs energy ΔG which takes a (accordingly high) negative value. Thereby, according to the invention, the groups are selected in a manner that the change of the Gibbs energy ΔG leads to a binding strengthening such that an improved separation selectivity results towards the substances to be separated off.

In general, an improved separation selectivity occurs if the ΔG value for the bond between the selected complementary groups of the produced sorbent and the second substrate (the target substance) is in a sufficient manner more negative (or becomes more negative) than the ΔG value between said sorbent and a substance to be separated off. In the chromatography, in this type, the substance to be separated off elutes earlier, said substance is weaklier bound. However, an improved separation selectivity occurs, if the substance to be separated off binds stronger than the second substrate (target substance) by way of the insertion of other complementary groups, thus due to the change of the ΔG value being associated therewith.

According to the invention, the target of the separation due to a sufficient separation selectivity is always achieved, if in the sorbent/substrate complex at least one complementary group more or stronger (such as in stereoisomers) participates in the bond with the second substrates than in the complex between the sorbent and the at least one substance to be separated off.

Examples for typical values of said interaction Gibbs energy ΔG (kJ/mole) being dependent from the solvent are
−4 to −6 for the ionic interaction, whereby the strength respectively the range of said interaction reciprocally decreases with the distance. An example for such an interaction is the interaction between a carboxylic acid and a quaternary amine hydrogen in water;
−1 for the ion/quadrupole interaction, whereby the strength respectively the range decreases with the third power of the distance. An example is the interaction between quaternary nitrogen in ammonium compounds and an arene group in water;
−1.75 for the disperse interaction (induced dipoles), whereby the range respectively the strength decreases with the sixth power of the distance. An example is the interaction between two arene groups in chloroform;
−4 to −6 for hydrogen bonds. An example is the interaction between two amide groups in chloroform. In carbon tetrachloride, the interaction energy between such groups is approximately −10;
−2.3 for the hydrophobic effect, as a result of the interaction between alkane and methylene radical in water.

If, according to the invention, in chloroform, hydantoins are bivalently bound to ammonium groups, ΔG values up to −22 kJ/mole are measured. In case of monovalently binding of a succinimide derivative, however, the ΔG values are averagely solely −9 kJ/mole. Thus, the difference of both ΔG values is approximately 13 kJ/mole, the corresponding value for the separation selectivity is approximately 200. Said data suggest hydrogen bonds and, predominantly, an entropic strengthening of the bivalent interaction.

In a given solvent system, for each type of non-covalent interaction and for each pair of first and second substrate (receptor/ligand), the distance-depending Gibbs energies can be differently composed of an enthalpy and an entropy contribution.

According to the invention, said individual contributions are determined by the analysis of the binding strength of a first substrate containing one, two, three, . . . n groups capable of binding, with, for example, a set of second substrates whose groups capable of binding are selected in a manner that conclusions are possible concerning a certain type of interaction. So, first substrates can be used which, preferably, contain amino, acetyl, benzyl, nitrophenyl and isopentyl residues, as well as combinations of two and three residues thereof. Then, the second substrates consist of derivatives of, preferably, alanine, aspartic acid and glutamic acid. Preferably, the N-terminated protective groups of said derivatives are either aliphatic or aromatic.

Preferably, the bond energies can be determined as k′-values from isocratic HPLC experiments. If, at the first substrate, the concentration of the groups capable of binding and the phase/volume ratio between the immobilized (stationary) phase and the mobile phase are known, the bond constant K_{A} can be determined from the k′-value, and, in turn, from said value the change of the Gibbs energy ΔG. For example, the enthalpy change ΔH and the entropy change ΔS can be microcalorimetrically determined or by way of temperature-dependent measurement of the equilibrium constant which also is denominated as van't Hoff plot. Subsequently, by way of comparison of the respective interaction energies between selected receptor variants and ligands, it is verifiable to what extent interaction contributions add, strengthen or weaken each other. It is self-evident that the methods for the determination of the binding are not restricted to the above mentioned ones. Besides, all common determination methods can be used, such as competitive assays, surface plasmon resonance or NMR titration. The determination of the interaction energies can be carried out in form of miniaturized assays and in parallel.

Under sterically favorable conditions, for the groups capable of binding, the parts of the Gibbs energy add each other. Consequently, the contribution to the bond constants multiply each other. Moreover, co-operative effects are possible contributing to the further binding strengthening. Also, under conditions being sterically less favorable, mostly an at least bivalently binding strengthening can be achieved. This is of high benefit for the practical application, because the binding strengthening for a suited choice of the residues capable of binding nearly completely results in an improved separation selectivity towards the substances to be separated (accompanying substances/by-products).

The term non-complementary means that groups can indeed interact with each other, however, said groups weaklier contribute to the non-covalent bond than complementary groups. Consequently, the binding strength between non-complementary groups is weaklier developed than the bond between complementary groups. According to the invention, groups not being complementary towards each other weaken the non-covalent bond that is formed between said groups, or weaken the respective entire binding site, or they are non-bonding. They are preferably characterized in that the contributions of the Gibbs energies of the individual groups for the non-covalent bond result in a change of the Gibbs energy ΔG that is zero or takes a positive value.

The term determination means a targeted selection, for example a targeted selection of groups capable of binding.

The at least one sorbent that is produced according to the new method can be used for the recognition of sorbent/substrate interactions. In particular, as method for the recognition, the new method is suited for the selectively binding of said substrate to the at least one said sorbent. As measure for the recognition, the binding strength can be used. In case of a sufficiently strong bond between sorbent and substrate, one obtains an information which groups of the substrate and which groups of the sorbent can bind each other.

If the groups of the substrate are unknown, in case of binding, one can conclude which groups capable of binding can exist in the substrate at the binding site.

However, it is also possible to separate molecular regions of a first substrate of unknown structure into suited components, for example epitopes, and to adjust said structure or a structure being complementary thereto by suitable arrangement of the components on the sorbent.

Thereby, the separation can be carried out both according to chemical, physical, or chemical-physical methods, for example by chemical degradation reactions or by ultrasonic, however, also by virtual experiments. For said virtual experiments, also computer-aided methods can be used by way of which information about the binding possibilities can be obtained that exist in the components of the substrates.

Starting point for the separation is that the set of all components capable of interaction and the number of the groups capable of binding is finite and limited, and, moreover, for a concrete problem, can be limited accordingly. From each arbitrarily selectable sub-set of such groups, one can produce arbitrary classes of combinations with m elements (m=2, 3, 4, . . . ), respectively. An example would be class 3 with all possible combinations of three groups capable of binding, respectively, from a selection n=5 with, for example, phenyl, alkyl, amino, carboxyl and amide groups.

In this manner, each protein can be separated into 20 components, thus the amino acids, from which, in turn, in a first approximation n=6 up to n=9 groups capable of binding are relevant for the non-covalent interaction with a second substrate. This reduction is accomplished thereby that the same group or an equivalent group capable of binding is contained in several amino acids, such as the hydroxyl, carboxyl and amide group, and also a basic function, if gradual gradings between lysine, arginine, tryptophan or histidine are not important.

In a comparable manner, the 8 isomeric ketohexoses or the 16 stereoisomeric aldohexoses and the pyranosides and furanosides derived therefrom can be employed as components that represent oligosaccharides.

This means that each arbitrarily unknown substrate consists of a countable amount of components which, in turn, contain a defined amount of groups capable of binding, respectively. The components and the groups capable of binding originates from the chemical knowledge and are, as a rule, known according to type and properties. This mainly applies if they can be assigned to the organic chemistry or to the complex chemistry.

Because one can synthesize in advance for each combination of the known components and the groups capable of binding libraries in arbitrary scope of sorbents being complementary and identical thereto, fundamentally each component from a molecular region or from a binding site of a first substrate of unknown structure can be included or can be involved in such a sorbent library. The same applies to the combinations of the groups capable of binding.

In the method according to the invention, also several sorbents can be obtained, thus a collection of sorbents. Now, one can contact a known or unknown second substrate being different from the first substrate and whose groups capable of binding are known, with said collection of sorbents and can determine the binding strength. Through this, one obtains an information how the components are arranged at the binding site of the second substrate, and how the spatial structure of the binding site is arranged. Thus, the novel method can also be used for the structure determination.

Furthermore, the novel method for the selectively binding of said substrate is extraordinarily valuable also for the development of agents, preferably for the development of drugs. It is generally known that the effectiveness of a drug is based thereon that it is bonded under physiological conditions to a natural receptor which, for example, can be a hormone or an enzyme. It is now possible to separate the binding site of the natural receptor in the manner described above, and to generate a collection of sorbents. Then, each sorbent from the collection of said sorbents contains defined components (parts or portions) of said binding sites. Preferably, thereby also the spatial arrangement of the components, further preferred the spatial arrangement of the components of the entire binding site, is imitated. If one now determines the binding strength of an arbitrary substrate, for example a drug, towards each of said synthetic receptor parts, from which now each represents another structural part of the natural receptor, one obtains an information from the binding data whether said substrate generally can well interact with the natural receptor, and, if yes, with which of the spatially arranged receptor groups, Then, by appropriate chemical modification, the substrate, thus the drug to be developed, can be optimized until the maximum binding to the receptor is given.

Preferably, the method is suited for isolating biopolymers that are unknown or that are only postulated for a certain function until now, preferably proteins or glycoproteins, and to validate said proteins or glycoproteins according to their properties.

In a comparable manner, it is conceivable to synthesize to peptides from phage displays, a sorbent structure that is complementary to oligonucleotides or to other matrices which can be used for the isolation of agent molecules directly from mixtures.

Conversely, by design of the structure parts being typical for agents on the sorbent surface, it is conceivable to bind from substrate mixtures the respectively corresponding substrate, and to characterize it. For example, such a substrate is a receptor.

In step (i), the selection of at least two different groups capable of binding a first synthetic or natural substrate to a sorbent, is carried out by determination of said groups from a synthetic first or natural first substrate. The determination of at least two different groups capable of binding a first synthetic or natural substrate to a sorbent can be carried out in any imaginable manner, i.e. that arbitrary groups can be selected by arbitrary methods, as long as these groups are capable of binding. In a preferred embodiment, the selection is carried out corresponding to the non-covalent interactions to be expected with the substrate.

In said embodiment of the invention, preferably, the determination according to step (i) comprises the separation of a synthetic or natural first substrate into at least two components having at least two groups capable of binding a sorbent.

In another embodiment, the invention envisions that the at least one first substrate is the same substrate as the at least second substrate and the respective at least two different groups capable of binding the second substrate are selected among such groups that are complementary to the groups which are determined in step (i).

Another embodiment of the invention is characterized in that the at least one first substrate is different from the at least one second substrate, and that the respective at least two different groups capable of binding the second substrate are selected among such groups that are complementary to the groups which are selected in step (i).

Another embodiment of the invention is also characterized in that the at least two groups capable of binding the at least one second substrate are selected among the groups that are determined according to step (i), i.e. the groups of the second substrate capable of binding are complementary to the corresponding groups of the first substrate.

Within the scope of the invention, in one embodiment, it is possible to separate in step (i) the synthetic or natural substrate only into two components having each one group capable of binding, whereby in step (ii) only one sorbent is obtained.

However, it is also possible to separate the synthetic or natural substrate into three components, whose pairwise combination results in three sorbents in step (ii).

In separating into four components, six sorbents are obtained by pairwise combination in step (ii).

However, it is also possible that in case of three different components besides the pairwise combination in step (ii), said three components can be applied together as a triplet onto a sorbent. Besides the above mentioned three sorbents, additionally a forth sorbent is obtained.

In an analogues manner, it is also possible that in case of four different components besides the pairwise combination in step (ii) that results in six sorbents, additionally four sorbents can be obtained which contain three different components, respectively, and another sorbent which contains all four components as a quartet.

Consequently, the invention is also characterized in that the determination of at least two groups capable of binding a sorbent from a synthetic or a natural first substrate in step (i) yields two components each having at least one group capable of binding the sorbent, and in step (ii) one sorbent is obtained; or the determination of at least two groups capable of binding a sorbent from a synthetic or natural first substrate in step (i) yields three components each having at least one group capable of binding the sorbent, and in step (ii) at least three sorbents are obtained; or the determination of at least two groups capable of binding a sorbent from a synthetic or natural first substrate in step (i) yields four components each having at least one group capable of binding the sorbent, and in step (ii) at least six sorbents are obtained.

Likewise, it is also conceivable to select from a larger number of i components n components and to combine therefrom multiplets from m groups capable of binding, respectively. For example, one can select from the set of the natural amino acids the components phenylalanine, tyrosine, isoleucine, aspartic acid, asparagine, serine, lysine, tryptophan and histidine (n=9), through which the most important types of non-covalent interaction can be covered. The combination of each m=4 different groups capable of binding from said selection yields 126 different variants of sorbents that also can be used in combinatorial manner or as an assay for binding purposes and binding studies.

Each of said m non-covalent interaction contributions provides for each individual sorbent a characteristical value for the total interaction with a substance to be bound. Said individual contributions of each group capable of binding (m=1) can be experimentally solvent-dependently determined for any substance to be bound within a range that can be neglected for the application. Likewise, one can obtain the measuring data for the doublet interactions with m=2, for the triplet interactions with m=3, etc.

Thereby, a comprehensive set of energy increments is obtained for the different forms and combinations of non-covalent interactions, then allowing the prediction of the binding strength between two arbitrary substrates or components. Thereby, also the fact is used that the different non-covalent interactions are dependent on solvent and pH. So, the hydrogen bond interactions have a strong influence in aprotically nonpolar organic solvents, but little influence in protically polar solvents or in water. With basic residues, carboxyl groups give strong ion bonds in organic solvents, however, as a rule, in water only a comparably lower entropydriven interaction is detected.

Exemplarily, said correlations are illustrated at hand of the binding of amino acid derivatives to different sorbents. Thereby, as already outlined, one can conclude from the k′-values of the chromatographical measurement to the bond constant K_{A} provided the concentration of the components that are attached to the sorbent or the groups capable of binding are known. Through this, a fast method is provided that can be used in parallel in order to obtain bond constants from substrates competing for the binding site, also if these are present in a complex mixture.

From the values of the bond constant and from the bond energies which can be obtained for the combinations of multivalent interactions, it is possible in the described manner to conclude to the type and to the number of the groups capable of binding of a structurally unknown substance to be bound, or to postulate the absence of other groups. So, conclusions can be made concerning the number of carboxyl groups, of basic groups or aliphatic or aromatic residues in a bound amino acid derivative or peptide.

Likewise, one can make conclusions about the structure-dependently estimated or the possible binding behavior between two substrates having an unknown structure, as soon as their groups capable of binding are known. This can apply to peptides or protein fragments, if one solely knows the composition of the amino acids.

Likewise, it is conceivable, to predict or to describe the binding behavior between two substrates of unknown structure, if said substrates have a stable spatial structure in the selected solvent system. Two proteins or glycoproteins with defined tertiary structure interacting with each other at least one binding site, will undergo interactions of similar strength or ranking with the members of a library of sorbents that are complementary to each other, respectively.

Another important application describes the manufacture of sorbents that represent a complete set of all combinations of groups capable of binding being complementary to a binding site at a protein or glycoprotein. Then, said library of sorbents is tested with a complete set of ligands which, for example, represents all combinations of two, three and four groups that are exactly capable of binding at the protein binding site. Then, those groups capable of binding are located at the sorbents that each have the strongest bond which, preferably, should be contained in an agent to be developed. It is self-evident that also the proteins can be bound to said sorbents having served as model for the complementary groups.

In an analogues manner, from the binding pattern of a cyclic peptide that is obtained by means of a phage assay, one can conclude to the binding site in the respective protein target. Moreover, it is conceivable to create by complementarily mapping of such a peptide a sorbent-supported matrix for the discovery of new agents having suited configuration and conformation corresponding to said peptide.

For this, said method can be used for the binding, characterization and validation of unknown protein targets and of binding sites for non-competitively or modulatorily acting agents. Furthermore, it is possible to realize flexible and instable agents, such as peptides, within rigid structures with satisfying administration possibility.

In all mentioned cases, the structure prognosis is made possible thereby that the substrates are contacted with a suited selection of sorbents and the binding data are measured. Thereby, for the deduction of a complementary substrate structure, missing or weak interactions are as important as a strong bond. If, for example, a substrate contains an amino acid, the bond to the sorbent containing the carboxyl groups will be higher for a characteristical amount than the bond of the same substrate to a sorbent carrying hydroxyl groups or even amino groups.

An essentially practical value of said approach is the exclusion of the majority of conceivable possibilities of the binding, whereby at least a limitation of the work to a further investigable number of possible binding combinations takes place. The same principle is used in the screening, in testing a substance mixture for substances having predetermined structure features that are contained therein. Thereby, the highly practical benefit is the achieved exclusion of the large majority of unusable substances without additional work.

Preferably, the dissection of the components is carried out in a manner that components are obtained that are in direct spatial proximity in the binding site of the natural or synthetic substrate. The spatial arrangement of the binding site can be characterized by dissection into two components by a linear arrangement of said components, for three components by a triangle and for four components by a (distorted) tetrahedron.

If said binding site is formed in a manner that in said binding site preferably three or four components exist with at least one group capable of binding, respectively, stereoisomeric substrates, as they exist for example in racemic mixtures, are, in general, differently strong bound.

Consequently, also stereoisomeric substrates can be differently strong bound according to the method according to the invention by way of the at least one sorbent according to the invention. This property can be taken for the agent development, because it is known that stereoisomeric compounds can have different physiological activity.

Thus, the novel method is a valuable method for the selective separation of one or more stereoisomeric compounds from a mixture of stereoisomeric compounds. For example, it can be used for the resolution of racemics.

As further stereoisomeric compounds which can be selectively bound, diastereomers, conformers, geometric isomers, such as cis and trans isomeric compounds, epimers, as well as anomers, such as α- and β-glycosidic sugars, can be mentioned.

However, not only stereoisomeric compound can be selectively bound by means of the new method, but also constitution isomers, that is compounds having the same elementary composition, in which, however, the elements are differently relatively arranged towards each other.

For example, it is conceivable to separate fused aromatic systems having the same empirical formula but differing in the type of the linkage of the carbon rings.

In applying at least two different groups capable of binding onto a carrier each of step (ii), respectively, according to the methods as described subsequently, in general it cannot be avoided that in at least one of the formed sorbents not only binding regions are generated, in which the desired at least two different groups capable of binding coexist in a statistical distribution, however, that also regions are generated, in which, in essential, only the same groups are present, or regions, in which said groups are enriched. However, such regions do not disturb the selective separation of said substrate because such a region in general binds weaklier than a region which contains the at least two different groups. Mostly, such a region essentially containing only one type of groups capable of binding, even repels said substrate. In particular, such a region is repellent if non-complementary groups are standing vis-á-vis each other.

Generally, all in all, non-complementary groups standing vis-á-vis each other will weaken the binding at the first and second substrate. Said effect already occurs with bivalent bonds. If, for example, as groups capable of binding, on the one hand, the carboxyl residue and, on the other hand, the amine residue as well as on the one hand, the phenyl residue, and, on the other hand, the fluorenyl residue were selected, each spatial arrangement is energetically relatively less favorable, in which at least one of the polar residues stands vis-á-vis a nonpolar residue. Because of the movable arrangement of the polymer chains, the second substrate to be bound at the sorbent will spontaneously attach in a manner that the maximum possible Gibbs energy is gained.

In general, one can express said facts in a way that, in the sorbent, a pair of complementary groups must stand vis-á-vis the pair of groups capable of binding. A bond between a sorbent and a ligand reaches its maximum strength if all involved groups are able to complementarily arrange each other in pairs or in multiplets, respectively.

Already in the bivalently matching of two substrates, the direction dependency becomes apparent. Said steric guidance will be considerably strengthened in changing to trivalent and tetravalent interactions. For a high yield of energetically optimal binding sites, one needs polymer derivatives with particularly high conformative movability. Thereby, copolymers are conceivable, in which between the bonded groups capable of interaction, sub-regions with highly conformative movability are integrated, for example alkyl chains.

The molar ratio respectively the local concentration ratio of the at least two different groups capable of binding that are applied onto the at least one sorbent, is extraordinarily important for the selectively binding of a substrate. Preferably, each group at the substrate to be bound must also find a group capable of binding at the sorbent.

Thus, preferably, the at least two different groups capable of binding are applied in a molar ratio optimally corresponding to the structural requirements of the substrate to be bound.

Preferably, the at least two different groups capable of binding which, preferably, are the same or are complementary to the groups of the first or second substrate, are applied onto the sorbent in a molar ratio as it also exists in the substrate to be bound, or as it exists in the copied first substrate. The thereby preferred used preparative methods are described beneath.

The synthetic or natural substrate of step (i) can have a low molecular weight, preferably a molecular weight below 1000 Da. Thereby, however, said substrates can also be oligomers or polymers, preferably biopolymers.

Preferably, one substrate has a low molecular weight and the other substrate is a biopolymer.

Preferably, the at least one sorbent capable of binding preferably biological substrates has one group capable of binding which is also responsible for the binding of structures that occur in the nature or for the binding of decisive parts of such structures, and which can interact with the substrate which, preferably is a biological substrate. In the following, the groups are also termed as receptors or receptor groups.

Preferably, the at least two groups capable of binding are parts of components or parts or fragments of substrates having functional groups. Thereby, here, in particular, enzyme groups, amino acid groups, peptide groups, sugar groups, amino sugar groups, sugar acid groups as well as oligosaccharide groups respectively derivatives thereof, as well as nucleosides and nucleotides are to be mentioned. Other suited substrates are pyrimidine bases and purine bases, such as cytosine, uracile, thymine, purine, adenine, guanine, ureic acid, hypoxanthine, 6-thiopurine, 6-thioguanine, xanthine.

Fragments of molecules are, for example, phenyl, phenol, or indole residues from phenyl alanine, tyrosine or tryptophan as well as hydroxyl, carboxyl, amino and amide groups. Solely, it is essential for the mentioned groups that the binding principle of a receptor with a substrate to be found in the nature is maintained or approximated, so that by way of the new method, for example, synthetic enzymes, binding domains of antibodies or other physiological epitopes, i.e. molecular regions, completed hosts, peptides, glycopeptides, epitopes of proteins, glycoproteins, as well as oligonucleotides can be applied.

Preferably, as amino acids the following acids have to be mentioned:
amino acids having aliphatic residues, such as glycine, alanine, valine, leucine, isoleucine;
amino acids having an aliphatic side chain which includes one or more hydroxyl groups, such as serine, threonine;
amino acids having an aromatic side chain, such as phenylalanine, tyrosine, tryptophan;
amino acids which include basic side chains, such as lysine, arginine, histidine;
amino acids which have acidic side chains, such as aspartic acid, glutamic acid;
amino acids which have amide side chains, such as asparagine, glutamine;
amino acids which have sulfur-containing side chains, such as cysteine, methionine;
modified amino acids, such as hydroxyproline, γ-carboxyl glutamate, O-phosphoserine;
derivatives of the amino acids mentioned above, or optionally of further amino acids, for example amino acids esterified on the carboxyl group or optionally the carboxyl groups with, for example, alkyl or aryl radicals which can be optionally suitably substituted.

Instead of the amino acid, also the use of one or more dipeptides or oligopeptides is conceivable, where, in particular, beta, gamma or other structurally isomeric amino acids and peptides derived therefrom, such as depsipeptides, can be used.

Thereby, it is also possible that with one component at least two different groups capable of binding are simultaneously inserted.

Consequently, the method according to the invention is also characterized in that one component carries at least two different groups capable of binding.

If more than four groups capable of binding should be attached to the same sorbent, then, a preferred embodiment consists therein to combinedly insert at least two of said groups capable of binding by way of an already completed component in defined spatial arrangement, respectively. Thereby, preferably, such groups capable of binding are attached in a component which were in proximity already in the first substrate.

It is self-evident that it is conceivable to successively or simultaneously insert several of such at least bivalent components into a sorbent, and furthermore to combine said components with monovalent components.

A simple example for a bivalent component is fluorenylmethoxycarbonyl glutamine, also termed as Fmoc glutamine. Here, the carboxyl group is used for the binding to the sorbent, whereby the amide radical is capable of the polarly binding of a ligand, and the fluorenyl group is responsible for the π-π interaction. In a similar matter, oligopeptides can be used, however, also comb-shaped derivatives of oligomers.

Preferably, the binding of said substrates to the at least one sorbent takes place via radicals or groups of amino sugars, sugars, nucleotides and nucleosides, as well as pyrimidine bases and purine bases that are present on the sorbent.

As a result, the invention is also characterized in that the at least two different groups capable of binding of the at least one sorbent are selected among groups which are part of amino acids, sugars, nucleotides, nucleosides, pyrimidine bases or purine bases.

In another embodiment, the at least two different groups capable of binding of the at least one second substrate are selected among groups which are part of amino acids, sugars, nucleotides, nucleosides, pyrimidine bases or purine bases.

By way of inserting further groups having natural or synthetic origin, in particular having synthetic origin, the capability of the non-covalently binding of the sorbent can be targetedly varied, in particular can be strengthened.

For example, amino acids that are provided with synthetic protective groups can be applied for the new method. For example, amino acids being protected with the fluorenyl residue can be applied. Besides the fluorenyl residue, also residues such as the anthracenyl or the naphthyl group can be applied. Through this, by formation of further non-covalent bonds between the aromatic rings of the protective groups and the binding groups of the substrates, a strengthening of the binding properties can be achieved. As further examples, nitrophenyl residues and oligofluorophenyl residues and other electron-rich and electron-poor aromatic systems which are able to form π-π interactions are mentioned.

Preferably, the sorbent of step (ii) comprises a carrier which can be built up from inorganic or organic materials or inorganic and organic materials. As carrier materials, all materials are suited which can be applied by suitable methods onto the at least two different groups from step (i).

In case where the carrier material is a solid, the surface thereof can be a plane surface, such as glass or metal plates, or also curved surfaces or surfaces being embedded into porous material, such as tubular or spongy surfaces, such as zeolithes, silica gel or cellulose beads. Furthermore, the carrier materials can be of natural or synthetic nature. Inter alia, for example, gelatine, collagen or agarose are mentioned. Also porous or non-porous resins as well as plastic or ceramic surfaces can be used.

However, it is also possible to use as carrier one or more liquids, preferably such ones having a high viscosity. Preferably, suited compounds are silicone oils having high viscosity.

Preferably, the respective at least two different groups of step (i) are present on the carrier in a form covalently bonded to a polymer.

Thereby, the term “polymer” embraces also compounds having a higher molecular weight which are characterized in the polymer chemistry as “oligomers”. Thereby, also a polymer as well as mixtures of polymers can be used.

Without wishing to be restricted to certain polymers, as possible polymers, inter alia the following polymers may be mentioned:
polysaccharides, e.g. cellulose, amylose and dextrans;
oligosaccharides, e.g. cyclodextrin;
chitosan;
polyvinyl alcohol, polythreonine, polyserine;
polyethylen imine, polyallyl amine, polyvinyl amine, polyvinyl imidazole, polyaniline, polypyrroles, polylysine;
poly(meth)acrylic acid(esters), polyitaconic acid; polyasparagine; polycysteine.

Likewise, not only homopolymers, but also copolymers and, in particular, block copolymers and random copolymers are principally suited to be employed in the present method. Here, copolymers having non-functionalized components such as co-styrene or co-ethylene, as well as copolymers such as co-pyrrolidone may be mentioned.

Said polymers have at least two groups that are the same or are different which can be covalently bonded to the polymer by way of the at least two different groups capable of binding from step (i).

Therefore, one embodiment of the invention is characterized in that the respective at least two different groups in step (ii) are covalently bonded to a polymer.

Preferred functional groups of the polymer having at least two identical or different functional groups which may be mentioned are, inter alia, OH groups, optionally substituted amine groups, SH groups, OSO₃H groups, SO₃H groups, OPO₃H₂ groups, OPO₃HR groups, PO₃H₂ groups, PO₃HR groups, COOH groups and mixtures of two or more thereof, where R preferably is an alkyl radical. Likewise, the polymers having at least two identical or different functional groups can also contain further polar groups, for example —CN.

Thereby, in one embodiment, it is possible in step (ii) to firstly insert the at least two different groups capable of binding into said polymer via the at least two identical or different functional groups, whereby a polymer is formed which is derivatized with said groups. Said derivatized polymer can then be applied onto the carrier.

The derivatization of the functionalized polymer with the at least two groups can be carried out according to known methods, both in homogeneous and heterogeneous phase.

The derivatization in heterogeneous phase can be carried out by means of solid phase reaction.

If the polymers having the at least two identical or different functional groups are derivatized in homogeneous liquid phase with said at least two different groups capable of binding, then, preferably, mixed-functional or, alternatively, pre-derivatized polymers are applied in order to achieve an optimal solubility. Examples of these which may be mentioned are, for example:
partially or completely silylated, alkylated or acylated cellulose;
polyvinyl acetate/polyvinyl alcohol;
polyvinyl ether/polyvinyl alcohol;
N-butylpolyvinyl amine/polyvinyl amine.

Likewise, polymer/copolymer mixtures can also be employed. All suitable polymer/copolymer mixtures can be employed here, for example mixtures of the polymers and copolymers already mentioned above, where, inter alia, the following are to be mentioned here, such as:
poly(acrylic acid-co-vinyl acetate);
poly(vinyl alcohol-co-ethylene);
poly(oxymethylene-co-ethylene);
modified polystyrenes e.g. copolymers of styrene with (meth)acrylic acid(esters);
polyvinyl pyrrolidone and its copolymers with poly(meth)acrylates.

Preferably, the polymer having at least two identical or different functional groups is reacted prior to the derivatization with the at least two different groups with an activating reagent. Such reagents and methods for the application thereof are, for example, described in the WO 00/32649.

For example, as activating reagents compounds can be used which are derived from the structure element of the succinimide, whereby the N-bonded hydrogen atom is replaced by a —OCO—Cl group. Such an example is the following compound:

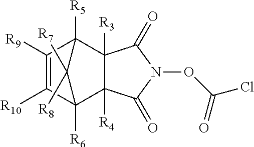

Thereby, R₃ to R₁₀ are preferably hydrogen, alkyl, aryl, cycloalkyl and heterocyclic residues. If the residues R₃ to R₁₀ are hydrogen, then, in the following, the compound is also termed as ONB-Cl.

If the polymer having at least two functional groups that are the same or are different, is reacted with an activating reagent, then said reaction product can be reacted with suited compounds having the groups that is required for the binding to said substrate.

It is also conceivable to react the polymer having two functional groups that are the same or are different, with a mixture of two or more suitable activating reagents. Said reagents can simultaneously be reacted with a polymer. Likewise, the two or more activating reagents can be subsequently reacted with a polymer.

Here, in principle, all compounds can be employed which can react with the activated polymer and which directly or indirectly result in the desired polymer which is then derivatized. For the derivatization, inter alia compounds can be employed having at least one nucleophilic group.

A further possibility is to react the activated polymer with an amino group-containing monohydric or polyhydric alcohol respectively mercaptan. If the polymer containing at least two functional groups is activated, for example with ONB-Cl, the monohydric or polyhydric alcohol containing the amino group or the monohydric or polyhydric mercaptan containing the amino group will selectively react with the amino group. The OH or SH groups thus inserted into the polymer can in turn be activated in a further step with, for example, one of the activating reagents described above, whereby chain extensions and branchings are facilitated, depending on the functionality of the alcohols or mercaptans originally employed.

In another embodiment, it is also possible to firstly react compounds each having at least one different group capable of binding with an activating reagent, and then to react the product obtained from said reaction with said polymer.

Preferably, activated derivatives of amino acids sugars, nucleotides, nucleosides, pyrimidine bases and purine bases are reacted with the polymer having at least two functional groups that are the same or are different. Thereby, in a preferred embodiment, in turn the compounds are activated with ONB-Cl or with a compound of said structural type.

Said reactions can be employed for polymer cross-linking, for polymer stabilization and for polymer branching.

Furthermore, said reactions make it possible to prepare polymer derivatives having a wide variety of spatial arrangements, and which, accordingly, can be used for a plurality of applications in which said spatial arrangement is of crucial importance.

Thus, for example, it is possible to realize arrangements which are constructed as hairy rods, comb polymers, nets, baskets, dishes, tubes, funnels or cages.

Thereby, the reactions can be carried out in aprotic-dipolar and/or polar-protic solvents or solvent mixtures, such as aqueous solvent mixtures. Depending on the polymer type to be reacted and the used activating reagent and/or compounds having the at least two different groups capable of binding, besides water different further solvents can be present in said solvent mixtures. Here, inter alia, solvents such as aprotic-dipolar solvents, such as DMSO, DMF, dimethylacetamide, N-methylpyrrolidone, tetrahydrofuran, or methyl-t-butylether can be employed.

The pH that is selected for said reactions, generally is in the range of from 4 to 14, preferably in the range of from 8 to 12 and, in particular, in the range of from 8 to 10. For the adjustment of a certain pH, suitable buffer solutions can be employed.

Via solvent and pH, the swelling and shrinking properties of the network can be targetedly adjusted, whereby by means of the network the access of the substrate to the sorbent can be influenced.

The derivatization degree of the polymer, that is the degree to which the functionalized polymer can be derivatized with the at least two groups capable of binding, can be influenced in a manner that the best possible interaction with the substrate is achieved.

A derivatization degree in the range of from 1 to 70% is preferred, more preferred in the range of from 3 to 60% and, in particular preferred in the range of from 5 to 50%.

Thereby, it is also possible that at least two of the functional groups that are the same or are different are derivatized so that they can interact as receptor groups with the substrate, and at least one functional group being not substrate-specific, and/or a monomer unit without functional group are situated between two of said derivatized groups, and whereby the functional groups are the same or are different of each other and are selected from the above-mentioned groups.

It also conceivable that the groups which still exist in non-derivatized form in the polymer contribute to the interaction with the substrate.

It also possible to use a derivative of a polymer having at least two functional groups that are the same or are different, in which another functional group being not substrate-specific is derivatized with an end-capping group.

By way of suitable choice of the end-capping group it is also possible to influence the solubility of the polymer derivative having the end-capping group or the end-capping groups and to adapt said derivatives to the requirements of possible subsequent reactions.

In principle, as end-capping group each group can be selected which renders a functional group inert or inert as far as possible towards interactions with the substrate. In this context, the term “inert” has the meaning that the interactions which the substrate undergoes with the receptor groups of the derivatized polymer are, compared to the interactions which the substrate undergoes with one or more functional groups that are derivatized with the end-capping group, so strong that the substrate essentially is only bound via receptor groups.

If it is desired to separate two or more different substrates via the interaction between substrate and receptor group, for example in a chromatographical method, there is no need for the end-capping group to completely render the functional group inert towards possible interactions, as described above. In this case, it is, for example sufficient if the end-capping group undergoes sufficiently weak or non-specific interactions with the two or more substrates to be separated which are not important for the separation method.

As an end-capping group, in principle any group can be used according to the prior art. Depending on the substrate, it is, for example, conceivable that as end-capping group a group is selected which is not an H-donor. Preferably

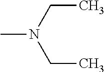

is employed here, particularly preferred is

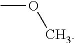

In a polymer having at least two functional groups that are the same or that are different, as receptor each of the above described residues can be inserted that is obtained by reaction of the polymer with at least two activated derivatizing reagents, each comprising at least one nucleophilic group, or by reaction of the activated polymer with at least two of such derivatizing reagents.

A derivative of a polymer having at least two functional groups that are the same or that are different is preferred, as described above, in which at least two receptors comprise residues of compounds or groups being responsible for the binding in compounds, whereby the compounds are selected from the group comprising amino acids, sugars, nucleotides, nucleosides, pyrimidine bases and purine bases.

In order to derivatize the polymer having the functional groups with the mentioned compounds, derivatives of said compounds or groups containing said compounds, or mixtures thereof, one can proceed according to the methods described above. So, it is conceivable to firstly carry out the reaction of, for example, an amino acid compound with a suited activating reagent, and then to react the reaction product with the polymer. It is likewise conceivable to firstly react the polymer with a suited activating reagent, and then with the amino acid. Naturally, it is also conceivable to directly admix the polymer, the amino acid and the activating reagent.

The insertion of residues of sugars, nucleotides, nucleosides, pyrimidine bases and purine bases, or of binding groups being contained in said compounds or mixtures thereof, is possible in an analogous manner.

Depending on the choice of the amino acids, sugars, nucleotides, nucleosides, pyrimidine bases and purine bases, or the respective residues or derivatives or the binding groups which are contained in said compounds, it may be necessary to possibly protect contained functional groups herein during the derivatization and/or the activation with protective groups. For this, all suited protective groups are possible which are known from the prior art. Depending on the later use of the polymers, after the derivatization, said protective groups can remain at the amino acid residue, the sugar residue, the nucleotide residue, the nucleoside residue, pyrimidine base residue or purine base residue, or they can be re-detached.

Instead of the amino acid, also the use of one or more oligopeptides is conceivable.

In order to optimize the interaction with the substrate, the liquid polymer derivative or the polymer derivative which is dissolved in a solvent or a solvent mixture can be deformed in the presence of the substrate which herein acts as template.

Thereby, for example, the deformation is carried out in a manner that, in a suitable solvent or solvent mixture, one mixes a derivatized polymer, as described above with the substrate, and allows the polymer to take one or more energy-favored conformations.

Thereby, it is also conceivable to mix and to deform a derivatized polymer with different substrates. Furthermore, it is also conceivable, if required, to mix and to deform different derivatized polymers with one or more different substrates.

It is also conceivable that the derivative of the polymer having at least two functional groups that are the same or that are different is deformed without template.

Subsequently to the deformation, the conformation of the polymer derivative which has been formed by way of the deformation in presence of the template can be fixed.

Here, it is also possible to apply the deformed polymer before the fixing onto a carrier.

In principle, for the fixing all conceivable methods are useable. In particular, here, the change of temperature, solvent, precipitation and cross-linking have to be mentioned. Preferably, the conformation is fixed by cross-linking.

Thereby, in essential, the carrier material and the form of the carrier are freely selectable, however, whereby the carrier material must be conditioned in a manner that the polymer can be permanently applied on the carrier. Preferably, the carrier material, after the derivatized has been applied, has no or only one or more non-specific interactions with the substances to be separated.

Dependent on the later field of application, it may be necessary that the carrier material is pressure-stable. In this context, the term “pressure-stable” has the meaning that the carrier material is dimensionally stable up to a pressure of 100 bar.

The above-mentioned materials can be used as carrier materials. Thereby, the shape of the carrier material can be adapted to the requirements of the method and is not restricted. For example, tablet-shaped, ball-shaped or strand-shaped carriers are possible.

The application onto the carrier material is largely freely selectable. For example, the application is possible by impregnation, by dunking the carrier into an appropriate polymer solution, by spraying the polymer onto the carrier or by concentrating the polymer by evaporation.

It is also possible to apply the derivatized polymer onto different suited carriers. It is likewise possible to apply two or more derivatized polymers being different of each other onto one or more suited carriers. In another embodiment of the method according to the invention, the derivatized, deformed and fixed polymer is processed to a porous material. Then, it simultaneously forms the carrier so that no additional carrier material is needed. Thereby, for example, beads, irregular particles, sponges, discs, strands or membranes can be obtained.

Thereby, one conformation can be fixed which was formed from one type of derivatized polymer. However, it is likewise conceivable that the conformation was formed by two or more types of derivatized polymers that are different of each other. Here, the term “different types of derivatized polymers” has the meaning that, for example, the polymers differ of each other with respect to the basic polymer, or the type of the activating reagent, or the type of the receptor groups which were inserted by derivatization, or the activation degree, or the derivatization degree, or a combination of two or more of these features.

Herein, for example, the cross-linking can be achieved thereby that two or more strands of derivatized polymers directly react with each other.

This can be achieved thereby that groups which were inserted by derivatization have such a nature that between said groups covalent and/or non-covalent bonds can be linked. Very general, it is conceivable that said covalent and/or non-covalent bonds are formed between groups that are attached to one polymer strand, and/or are formed between groups that are attached to two or more polymer strands, so that by way of the cross-linking two or more polymer strands can be linked via one or several sites with each other.

Likewise, it is also conceivable to apply for the cross-linking one or more suited cross-linking reagents, by means of which, as described above, groups can be cross-linked in a covalent and/or non-covalent manner within a polymer strand, and/or groups which are attached to several strands of optionally differently derivatized polymers.

In principle, as cross-linking reagents all suited compounds can be used known from the prior art. So, for example, the cross-linking can be carried out in covalent-reversible manner, in covalent-irreversible manner or in non-covalent manner, whereby in case of cross-linking in non-covalent manner, for example, cross-linkings via ionic interactions or via charge/transfer interactions have to be mentioned.

As cross-linking reagents which can lead to covalent-irreversibly cross-linking, inter alia, twofold or manifold functional compounds, as for example diols, diamines or dicarboxylic acids have to be mentioned. Thereby, for example, bivalent cross-linkers are reacted with the activated polymer derivative, or the at least bivalent activated cross-linking reagent is reacted with the non-activated polymer derivative.

A covalent-reversible cross-linkage can be realized, for example, by linking a sulfur-sulfur bond to a disulfide bridge between two groups that are attached to one or two polymer strands.

Cross-linking via a ionic interaction can take place, for example, via two radicals of which one has a quarternary ammonium ion as a structural unit, and the other has, for example, as a structural unit
or —COO⁻ —SO₃⁻

A cross-linkage via hydrogen bonds can be formed, for example, between two complementary base pairs, for example via the following structure

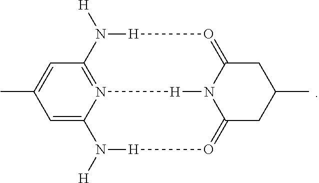

Very generally, polymers to be non-covalently cross-linked can be built up with respect to the cross-linking sites in a complementary manner, whereby structural units being complementary to one another are, for example, acid/triamine or uracile/melamine. Likewise, in a non-covalent cross-linkage, the cross-linking reagent can be complementary to the cross-linking sites on the polymer strand. An example is an amine group on the polymer strand and a dicarboxylic acid as a cross-linking reagent.

An amide bond towards the amino groups of the polymer can be produced from is the carboxylate by means of the coupling reagents which are known from the peptide chemistry. In the same manner, a carboxyl group that is covalently bonded at the polymer, is cross-linked with the amino groups of the polyvinyl amine, or vice versa, a bonded amino group is cross-linked with a carboxyl group, for example from polyacrylate.

Essentially, the cross-linking degree can be arbitrarily selected and, for example, can be tailored to the subsequently described application fields.

In step (ii), the reaction of the at least two different groups capable of binding with the polymer having at least two groups can also be carried out in heterogeneous phase, i.e. at the solid surface of the polymer. Advantageously, said polymer is suspended in a solvent having only a low solution power for the applied polymer.

For the derivatization of the polymer as well as for the application of the obtained polymer onto the carrier, the above described activating and derivatization steps as well as cross-linking methods and coating methods can be applied.

On the other hand, it is also possible to use as a carrier the polymer which is preferably derivatized in heterogeneous phase without further carrier material.

In another embodiment, preferably the above-described derivatized polymers that are synthesized in homogeneous or heterogeneous phase can be applied in steps onto the carrier. For this, in at least one step, at least one layer of the at least one polymer is bound to the carrier material and in at least one further step at least one further layer of the at least one polymer is applied onto the at least one polymer layer which is bound to the carrier material. Suited methods are described in the WO 01/38009.

Here, the stepwise application of the at least one polymer can be realized according to all suited methods which ensure that per step at least one layer of the polymer is applied so that a layered polymer structure is applied onto the carrier material.

In a first embodiment of said method, in at least one step in which the at least one layer of the at least one polymer is bound to the carrier, a solution of the at least one polymer is contacted with the carrier material under reaction conditions in which the at least one polymer is not bound on the carrier material, and subsequently the reaction conditions are varied in a manner that the at least one polymer is bound to the carrier material, or, in a second embodiment, a solution of the at least one polymer is contacted with the carrier material under reaction conditions in which the solution of the at least one polymer is present under theta conditions.

Here, the solution which is contacted with the carrier material according to the first embodiment can have one or more solvents, whereby the at least one polymer is dissolved in the solvent or the solvent mixture, or can also be colloidally dissolved or also suspended, for example, in form of a nano suspension.

Then, the reaction conditions are selected in a manner that by contacting the solution with the carrier material firstly no binding of the at least one polymer to the carrier material takes place. For example, said reaction conditions are adapted by one or more suited solvents. For this, preferably solvents are applied in which the at least polymer is so well dissolvable that the binding to the carrier material is stopped.

In the meaning of the present invention, the term “the polymer is not bound to the carrier material” has the meaning that by means of the measurement of the partition coefficient essentially no binding can be detected.

Likewise, said reaction conditions can be achieved by suitable choice of the temperature, whereby, for example, the solution is contacted with the carrier material at temperatures so high that the binding of the at least one polymer to the carrier material is stopped.

Furthermore, said reaction conditions can be achieved by suitable adjustment of the pH of the polymer solution in case that the binding of the at least one polymer to the carrier material is pH-dependent.

Likewise, it is also conceivable to firstly prevent the binding of the at least one polymer to the carrier material by suited combination of two or more of these methods.

By means of this specific type of the reaction guidance, inter alia it is achieved that reaction conditions can be avoided, among which the at least one polymer being contained in the solution precipitates.

Concerning the contacting of the solution of the at least one polymer with the at least one carrier material, in principle all suited process conditions are conceivable.

So, for example, it is possible to contact a solution containing the at least one polymer with the carrier material. It is likewise conceivable to firstly contact the carrier material with the at least one solvent and then to insert into the at least one solvent the at least one polymer. It is likewise possible to firstly contact the carrier material with at least one solvent and then to add a solvent comprising the at least one polymer. If two or more polymers are applied, it is conceivable to separately dissolve each polymer or together with one or more other polymers in one solvent or solvent mixture, respectively, and to combinedly or separately contact the individual solutions of which each comprises at least one polymer, with the carrier material that already is dissolved or is colloidally dissolved or is suspended in at least one solvent.

In principle, the already above-described carrier materials are suited, on which the at least one polymer can be applied by binding. If two or more polymers are applied that are different of each other, it is sufficient if one of the polymers can be applied onto the carrier material. It is also conceivable that two or more different polymers can be applied onto the carrier material by binding.

If two or more polymers being different from one another and two or more carrier materials being different from one another are applied, then, inter alia, it is conceivable that all polymers are applied to all carrier materials. It is likewise conceivable that one or more polymers can be applied onto one or more carrier materials, and that one or more polymers being different therefrom can be applied on one or more carrier materials being different therefrom.

Furthermore, further polymers and compounds, such as the generally known additives, can be applied, whereby the binding of the polymer to the carrier material can also be accomplished by way of other interactions and/or methods. Furthermore, the polymers or/and compounds being present in the solution cannot be applied onto the carrier, and, for example, can remain in the solution. Inter alia, it is conceivable that in further step at least one of said polymers is applied, for example onto a carrier material that is contacted with the solution comprising said polymer prior to said further step.

According to the first embodiment, after the contacting, the reaction conditions are changed such that now the binding of the at least one polymer to the carrier takes place. As described above, it is conceivable that in case that two or more different polymers or/and two or more different carrier materials are applied, a polymer is bound to one carrier material.

Concerning the variation of the reaction conditions, all changes are conceivable being suited to allow the binding of the at least one polymer to the carrier material.

In case that the binding is temperature-dependent, for example it is conceivable, either to increase or to decrease the temperature, whichever change favors the binding. In a likewise preferred embodiment, the composition of the solution containing the at least one polymer is changed, or said solution is slowly concentrated.

Concerning the change of the composition of the solution containing the at least one polymer, in principle all methods are conceivable being suited to allow the binding by way of said change.

In a preferred embodiment, another solvent is added to the solution in which the at least one polymer is contained which has worse dissolving properties with respect to the at least one polymer.

In another embodiment, the composition of the solution is changed such that at to least one acidic or at least one basic compound or a mixture of two or more thereof is added by means of which the pH of the solution is changed in a way that the binding of the at least one polymer is made possible. It is self-evident to add one or more buffer solutions by means of which the pH of the solution is changed in a way that the binding of the at least one polymer is made possible.

Further, suitable compounds, such as salts comprising, for example, metal cations or suited organic compounds, can be added by way of which the binding of one of the polymers takes place.

The solution containing the at least one polymer can also be concentrated such that the concentration of the at least one polymer to be bound to the carrier material largely remains constant in the solution. Said concentration of the solution takes place by means of an appropriately slow process guidance by means of which the polymer concentration is largely kept constant.

Further, two or more of the above mentioned methods can be combined in a suited manner under inclusion of the change of the temperature. So, for example, it is conceivable to vary the composition of the solution as described above and to supportedly slowly concentrate the solution or/and to suitedly vary the temperature.

Dependent on the selected reaction conditions, it is conceivable that one polymer or more polymers that are different of each other are applied onto the carrier material. Inter alia, it is conceivable to select the reaction conditions such that two or more polymers that are different of each other are simultaneously applied onto the carrier material, whereby one layer is generated on the carrier material which comprises the two or more polymers that are different of each other. If two or more carrier materials that are different of each other are used, it is conceivable to apply on each carrier material one layer of a polymer which can comprise a polymer or two or more polymers that are different of each other.

Furthermore, it is also possible that in one step two or more layers of at least one polymer are applied onto the carrier material, whereby the first layer of the polymer is bound to the carrier material, the second layer of the polymer is bound to the first layer, and, optionally, each further layer of the polymer is bound to the respective proceeding layer. Thereby, in principle, each layer can comprise one polymer type or two or more polymers being different of each other.

Furthermore, according to the second embodiment, a solution of the at least one polymer can be contacted with the carrier material under reaction conditions in which the solution of the at least one polymer is present under theta conditions. With respect to said embodiment, the application of the at least one polymer to the carrier material in particular takes place during the contacting of the solution with the carrier material.

According to the method described before, preferably in first step a layer of at least one polymer is applied to the carrier material, and, in a second step, onto said first layer a second layer, and in a third step, onto the second layer optionally a third layer, and so on. With respect to suited methods of the application, reference is made to the above discussion.

The term “binding of the polymer to the carrier” embraces all covalent-reversible, covalent-irreversible and non-covalent interactions by means of which at least one polymer can interact with the carrier material or/and with a polymer layer optionally already being applied onto the carrier material, or a polymer layer optionally already being applied onto a polymer layer.

Accordingly, essentially all polymers can be applied which, for example are capable of forming such non-covalent interactions. Here, inter alia, it is conceivable that at least one functional group by means of which the polymer forms at least one of said interactions, is in the polymer strand itself or/and in at least one side chain of the polymer strand.

However, for example, interaction can take place by means of hydrocarbon chains and further structure units via which the van der Waals interactions can be built up.

With respect to the covalent-reversible interaction, inter alia, exemplarily the binding via disulfide bridges or via unstable esters or imines mentioned, such as Schiff's bases or enamines.

In another embodiment, all polymers or/and co-polymers described above or mixtures thereof can also be applied onto the carrier in a non-derivatized form, as long as it is ensured that, as described above, they can form covalent or/and non-covalent interactions to at least one carrier material.

For the derivatization of the polymer which is applied onto the carrier, the activation and derivatization steps described before can be used, possibly followed by cross-linking steps as described in the WO 00/32649 and WO 00/78825.

In said embodiment, the method according to the invention is characterized thereby that before the covalently binding of the at least two different groups to the polymer having at least two functional groups that are the same or that are different, said polymer is applied onto a carrier.

In another particular embodiment of the method, the polymer having at least two functional groups that are the same or that are different, can also be directly produced by polymerization or polycondensation of at least two identically or differently functionalized monomers.

Thereby, preferably, olefinic unsaturated monomers which preferably contain OH groups, optionally substituted amine groups, SH groups, OSO₃H groups, SO₃H groups, OPO₃H₂ groups, PO₃H₂ groups, PO₃HR groups, COOH groups and mixtures of two or more thereof, wherein R preferably has the meaning of an alkyl radical, can be polymerized with one other in presence of the carrier material according to the known methods. Also, the monomers can contain further polar groups, as for example —CN. Further suited monomers are, for example, ethylene imine, allyl amine or vinyl pyrrolidone.

Preferably, as polymerization techniques, the emulsion polymerization, suspension polymerization, dispersion polymerization and precipitation polymerization are mentioned, whereby the polymerization is carried out in presence of the carrier or the carrier material. The polymerization can be initiated by means of the common methods, for example by radical starters such as azo compounds or peroxides, by means of cationic or anionic starters or by means of energy-rich radiation.

In one embodiment, it is possible carrying out the polymerization such that no reaction takes place between the created polymer chains and the surface of the carrier. Preferably, said embodiment is used, if as at least one of the two monomers a hydrophilic monomer is applied, such as ethylene imine, allyl amine or vinyl pyrrolidone. In presence of a hydrophilic carrier, such as silica gel, normally the produced polymer is strongly adsorbed on the carrier surface.

For increasing the stability of the coated carrier, the polymer can also be cross-linked with the carrier. Preferably, this is achieved by heating, whereby functional groups of the firstly adsorbed polymer react with the carrier respectively functional groups of the carrier react with the polymer, whereby the binding takes place.

However, it is also possible carrying out the (co)polymerization such that the polymer is directly chemically bound on the surface of the carrier. Said embodiment is preferred, if particularly stable coated carriers are to be produced. For this, the carrier can be provided with groups which react under the polymerization conditions with the polymer chains being formed on the surface of the carrier. However, it is also possible that functional groups of the polymer react with the surface of the carrier. If silica gel is used as carrier material, for example, silicol groups that are present on the surface of the silica gel can take part in the polymerization of the at least two functionalized monomers, whereby carrier and polymer are coupled with each other. It is also possible, for example, to attach vinyl silanes to the surface of the carrier, whose vinyl groups take part in the copolymerization of the at least two identically or differently functionalized monomers.

For the further increasing of the stability of the formed stationary phase, the polymerization of the two identically or differently functionalized monomers can also be carried out in presence of one or more cross-linking reagents. Cross-linking reagents are, for example, bifunctional compounds, such as divinyl benzene or ethylene glycol diacrylate.

Also, at least two identically or differently functionalized monomer components which, preferably, have the groups mentioned before, can be polycondensated with one other in presence of the carrier material according to the known methods. Thereby, also the methods and reagents based on ONB-Cl can be applied as described in WO 00/32649 and WO 00/78825.

Preferably, the obtained functionalized polycondensates can be of the polyphenylene, polyester, polyamide, polyether, polyether ketone, polyether sulfone, polyurethane, or polysiloxyl silane type. In this reaction type, also mixed polycondensates can be produced. Thereby, the polycondensation can be carried out in solution as well as in the melt.

Preferably, polycondensates of the polyester type are used. For increasing the stability, these can be further cross-linked by means of addition of further polyfunctional compounds, such as polyvalent alcohols, such as trimethylolpropane, pentaerythrol, or sugar. Also, the cross-linking via polyfunctional isocyanates is possible, provided that said polyesters have groups which react with the isocyanate groups. For example, hydroxyl groups-containing polyesters can be reacted with polyisocyanates, whereby the polyester/urethane units are incorporated.

For example, the obtained coated carrier material can be isolated by filtering the reaction mixture which is obtained in the polymerization or polycondensation, and can be purified by rinsing with a suited solvent from polymer particles or polycondensation particles which are not bound on the surface of the carrier material.

Accordingly, the method according to the invention is characterized thereby that the polymer having at least two functional groups that are the same or that are different is directly produced on the carrier by polymerization or polycondensation of at least two identically or differently functionalized monomers.

It is also possible to carry out the before-described polymerization that leads to the coating of the carrier analogously to the known “imprinting technique” in presence of the substrate which is to be recognized later. In the language use of said technique, for the term substrate frequently also the term template is used.

A requirement for said polymerization is that the monomers having the at least two identically or differently functionalized monomers have already the groups capable of binding. Thereby, preferably, each of said monomers has one of said groups, whereby the groups are different.

However, it is also possible applying monomers already having at least two different groups capable of binding.

Preferably, the polymerization is carried out in presence of substances that form pores.

For carrying out the polymerization, the above-described polymerization techniques can be used.

After unhinging or rinsing out the substrate with suited solvents, in step (ii) at least one sorbent is obtained with a pre-formed interaction space for the substrate.

Preferably, for said embodiment, the monomers to be used for the polymerization are selected such that the polymer that is formed on the carrier has a scaffold as rigid and as highly cross-linked as possible, so that the interaction space is as stable as possible. So, preferably, as at least one of the functionalized monomers, acrylic acid or methacrylic acid or derivatives or mixtures thereof are employed which, as is generally known, allow the production of polymers or copolymers with high glass transition temperatures. Particularly suited monomers are, for example, methacrylic acid and ethylene glycol dimethacrylate.

Another example is the polymerization of methacrylic acid with hydroxethylacrylate, whereby a polymer is obtained having carboxyl and hydroxyl groups capable of binding.

However, it is also possible carrying out the above-described polycondensation which leads to the coating of the carrier in presence of the substrate to be recognized later, whereby as monomers such compounds are used which already have different groups capable of binding. Preferably, each monomer has one of said groups, whereby the groups are different.

However, it is also possible to employ monomers which already have at least two different groups capable of binding.

After unhinging or rinsing out the substrate with suited solvents, in step (ii) at least one sorbent is obtained with a pre-formed interaction space for the substrate.

Accordingly, said embodiment is also characterized in that the polymer is directly produced on the carrier by means of polymerization or polycondensation of at least one monomer having at least two different groups capable of binding, or of at least two monomers each having at least one group capable of binding, whereby said groups are different, and the polymerization or polycondensation takes place in presence of the substrate to be bound later.

Preferably, in the embodiments in which the polymerization or polycondensation of said monomers is directly carried out in presence of the carrier, the polycondensation or polymerization is carried out in presence of at least a second or third monomer having no group capable of binding. Thereby, the at least one second or third monomer has the function of a spacer.

It is not necessarily required that the at least two different groups needed for the binding of the at least bivalent substrate to the at least one sorbent are bound to a polymer. It is also possible to directly immobilize in step (ii) the groups on the surface of the carrier without the use of a polymer.

Preferably, the immobilization is directly carried out on the carrier, if said carrier is built up from an inorganic material. Preferably, inorganic materials are silica gel or alumina.

Preferably, the immobilization is carried out by means of activating and/or silanization reagents. The linkage to the surface of the carrier can also be carried out by using a spacer.

Preferably, as activating reagents, the reagents described in the WO 00/32648 can be applied.

Preferably, silanization reagents also comprise such silicon compounds which can perform a hydrosylilation reaction.

Preferably, as silanization reagents halosilanes are applied, preferably chlorosilanes, alkoxysilanes and silazanes.

Here, in one embodiment, a compound having the group needed for the binding of the substrate, can firstly be reacted with a suited silicon compound. Subsequently, the product can be immobilized by way of hydroxyl groups being on the surface on the carrier under formation of a covalent oxygen/silicon bond. For example, alkyl radicals which optionally can be substituted, for example with amino, urea, ether, amide and carbamate groups, can such be immobilized on the surface by using alkylated silanes

For example, it is possible in this manner to immobilize on the surface of the carrier the 3-aminopropyl radical via a silicon atom. Then, the amino groups can further be reacted, for example with acid chlorides to amides. Aliphatic, however, preferably aromatic acid chlorides can be used, as well as activated components, in particular ONB-activated components as described in the WO 00/32649 and WO 00/78825.

Examples for silicon compounds by means of which alkyl radicals can be applied onto the carrier, are methyltrichlorosilane and octyltrichlorosilane, by means of which relatively short-chain respectively medium-chain alkyl radicals can be inserted, as well as octadecyltrichlorosilane, docosyltrichlorosilane and tricontyl-trichlorosilane, by means of which relatively long chains can be inserted. For example, the insertion of an alkyl radical containing an amino group is possible with 3-aminopropyltriethoxysilane.

Further, the use of silyl glycidyl ethers is possible which, after hydrolysis, form diols which are also termed as diol phases.

On the other hand, it is also possible to firstly react the surface of the carrier with a silicon compound having another functional group or more functional groups. Subsequently, the groups that were selected or determined for the binding which are to be immobilized on the carrier, can be inserted by means of suited compounds via the one or more functional groups.

For example, for the application onto the surface of the carrier, silicon compounds can be used still having a double bond. The groups which are intended for binding can be inserted via said double bond. Examples for suited silicon compounds are vinylsilane or (meth)acryloxypropyltrimethoxysilane.

The described methods can also be used in combination.

Optionally, the coupling of the groups being intended for binding can also take place via a spacer, whereby, preferably, a short-chain carbon chain is incorporated between the group to be immobilized and the carrier. Preferably, the linkage of carrier and group to be immobilized can take place by means of suited carbodiimides, such as dicyclocarbodiimide, diisopropyl carbodiimide, N-cyclohexyl-N′-2-(N-methylmorpholino)-ethyl carbodiimide-p-toluene sulfonate, N-ethyl-N′-(3-dimethylaminopropyl)carbodiimide-hydrochloride, chloroformiates, carbonyl diimidazoles, or diisocyanates, such as hexamethylene diisocyanate. Also, homotelomeric or heterotelomeric polyethylene glycols can be used.

In using a spacer, preferably a brush-formed phase is created in which the at least two different groups capable of binding are preferably bound either at the end of the spacer and/or are laterally bound at the spacer.

Accordingly, said embodiment is also characterized in that in step (ii) the at least two different groups capable of binding with a second substrate are applied onto a carrier by means of a reagent which is selected from the group comprising activating reagents, silanization reagents and spacer, or mixtures of two or more of said reagents.

It has proven to be unfavorable for the substrate-specific binding to apply sorbents having as at least two different groups capable of binding the groups which are described in the prior art, that is to say hydroxyl groups from the silica gel scaffold respectively silicol groups and alkyl groups which are incorporated via the silanization reagent. Thus, a combination of the groups hydroxyl, silicol and alkyl or hydroxyl and alkyl or silicol and alkyl is excluded from the invention, whereby the groups are immobilized at the silica gel, respectively.

Particularly suited groups in the meaning of the invention are on the other hand groups such as the phenyl, the hydroxyphenyl, the carboxyl, the amine and the amide residue as well as the hydroxyl, indole, imidazole and guanidine residue. Preferably, said residues are bound at the surface of the carrier via a spacer under formation of a brush-shaped phase.

Accordingly, a particularly preferred embodiment is characterized in that in step (ii) the at least two different groups capable of binding with a second substrate are selected from the group consisting of phenyl, hydroxyphenyl, carboxyl, amine, amide, hydroxyl, indole, imidazole and guanidine residues.

Preferably, the at least one sorbent produced according to the preceding methods, can be processed according to the common methods to foils, films, micro titer plates, or nano beads. Preferably, the at least one sorbent of step (ii) is produced and used in nano formate.

The substrate to be bound respectively the substrate to be selectively bound from a substrate mixture is now contacted in step (iii) with the at least one sorbent. Thereby, the substrate or the substrate mixture can be present in solid phase, liquid phase or gaseous phase, or also in mixtures of two or more of said phases.

Preferably, substrate respectively substrate mixture are in liquid phase. Thereby, solutions as well as suspensions or dispersions of the substrate respectively the substrate mixture are employable. As liquids, both water and organic solvents, mixtures of organic solvents and mixtures comprising water and organic solvents can be used. In all cases, buffers, salts, acids, bases or modifiers, such as ion-pair reagents can be present in the liquid in an arbitrary concentration. Preferably, the concentration is of from 10 mmolar to 2 molar related to one liter of liquid. Preferably, the substrate to be bound is present in aqueous form, for example as body liquid.

For the testing of the binding respectively of the binding behavior of the substrate to the sorbent, the known methods and methods can be used. Preferably, the bond between sorbent and substrate is the non-covalent bond.

Preferably, the interactions which are described above are non-covalent bonds.

However, it is also possible that the at least one substrate is covalent-reversibly or covalent-irreversibly bonded to the at least one sorbent.

Preferably, in step (iv), for the testing of the binding strength of the at least one second substrate to the at least one sorbent of step (iii), chromatographical methods and interpretation methods are suited. In particular, said methods are column chromatographical methods, for example the known HPLC method. For this, the at least one sorbent is used as stationary phase of the column. From the sequence of the eluted substrates, the binding strength thereof to the respectively used sorbent can be directly concluded. The strongest bound substrate is eluted as last substrate.

It is possible carrying out front analysis in which diluted solutions of the substrate mixtures to be separated are continuously applied onto the stationary phase. The strongest bound substrate can be distinguished from the substrates that are less strongly bound in this way, because the latter ones firstly arrive in the eluate.

However, also the known elution techniques can be carried out, wherein relatively concentrated solutions of the substrate mixture are applied onto the column head and are then eluted with an eluent. The weakly bound substrates firstly arrive in the eluate. The strongest bound substrate may, as the case may, be also desorbed from the sorbent by using an eluent which elutes stronger.

Preferably, also the micro calorimetry can be employed. Here, the adsorption heat is measured which is released during the binding of the substrate to the sorbent.

Another method that advantageously can be applied is the surface plasmon resonance method, in which the resonance frequency of excitable electrons is determined which is dependent on the physical properties of the barrier layer of substrate and sorbent, thus also is dependent on the binding strength.

Preferably, also as test method fluorescence labeling may be used, whereby the substrates that are labeled with a fluorescent dye only then fluoresce if they interact with the complementary receptor.

Another method is the enzyme linked immunosorbent assay method (Elisa), in which, for example antigens that are bound to the sorbent, can be detected by treatment with immunoreagents. Also competitive and non-competitive assays are useable, among them are radio assays.

Accordingly, said embodiment of the invention is characterized in that in step (iv) for the testing of the binding strength of the substrate to the sorbent a method is used selected from the group comprising chromatography, micro calorimetry, surface plasmon resonance, fluorescence labeling, competitive and non-competitive assays including radio assay, and Elisa.

From the binding strength, an information can be obtained which of the sorbents respectively which of the groups being applied thereto are responsible for the binding of the substrate. Thus, said method allows to isolate, to identify and to characterize said substrate. Thus, the validation of function and properties of the substrate is possible.

Accordingly, the method for the selectively binding of said substrate is also characterized in that it additionally comprises the step (v):
(v) isolating the at least one second substrate.

Furthermore, the method for the selectively binding of said substrate is also thereby characterized that it additionally comprises the step (vi):
(vi) characterizing and identifying the at least one second substrate.

In particular, the sorbents produced according to the novel method are suited for to the selectively binding of natural substrates or natural agents as well as of synthetic agents. It is common for said substrates and agents that they have a pharmacophore, thus a spatial arrangement of groups forming the basis for the biological effect in living organisms. The pharmacophore attaches the agent to the binding pocket of the natural receptor. The pharmacophore is attached to a frame which, in the English literature is also termed as scaffold.

Preferably, natural substrates and agents comprise amino acids, oligopeptides, nucleotides, nucleosides, proteins, glycoproteins, antigens, antigen determinants, antibodies, carbohydrates, enzymes, co-enzymes, ferments, hormones, alkaloids, glycosides, steroids, vitamins, metabolites, viruses, microorganisms, substances contained in vegetable and animal tissue, cells, cell fragments, cell compartments, cell disruptions, lectins, flavylium compounds, flavones, and isoflavones.

In the context of the invention, it is of particular interest to dissect natural receptors and enzymes or other proteins with pharmacological activity, to generate with their aid a collection of sorbents according to the invention and to use said sorbents according to the invention. Preferably, said receptors are intracellular or membrane-located proteins which can bind synthetic or natural agents.

Intracellular receptors can be obtained from cytoplasm and from cell nuclei. Such receptors respectively sorbents having at least two binding groups of said receptors can be used for the selectively binding of steroid hormones, such as glucocorticoids, mineralocorticoids, androgens, estrogens, gestagens, vitamin D hormones, as well as of retinoids or thyroid hormones.

Membrane-located receptors, the groups of which can be applied onto sorbents according to the invention, are guanine/nucleotide/protein-coupled receptors, ion channel receptors and enzyme-associated receptors.

In particular, for the medical therapy, among the group of guanine/nucleotide/protein-coupled receptors are the important neurotransmitter receptors, such as adenine receptors and adrenergic receptors, ATP-(P2Y) receptors, dopamine receptors, GABA_{B} receptors, (metabotropic) glutamate receptors, histamine receptors, muscarine receptors, opioid receptors, and seretonine receptors. Also hormone receptors and mediator receptors, for example from adiuretine, glycogen, somatostatine and prostaglandins are among said group.

Ion channel receptors comprise ATP-(P2X) receptors, GABA_{B} receptors, (ionotropic) glutamate receptors, glycine receptors, 5-HT₃ receptors, and nicotine receptors.

Among enzyme-associated receptors are receptors with tyrosine kinase activity, receptors with associated tyrosine kinases, with guanylate cyclase activity and receptor/serine/threonine kinases.

Preferably, synthetic agents comprise pharmaceuticals and plant protective agents.

For example, pharmaceuticals are substances having influence on the nervous system (psychotropics, barbiturates, analeptics, analgesics, local and common anaesthetics, muscle relaxants, anticonvulsants, antiparkinsonian agents, antimetics, ganglial acting agents, sympathic acting agents, parasympathic acting agents); having influence on the hormone system (hypothalamus, hypophysis, thyroid, parathyroid and renal hormones, thymic hormones, agents influencing the endocrine part of the pancreas, of the adrenals, of the gonads); having influence on mediators (histamine, serotonine, eicosanoids, platelet-activating factors, kinines); having influence on the cardio-vascular system; having influence on the respiratory tract (antiasthmatics, antitussives, expetorants, surfactants); having influence on the gastrointestinal tract (digestion enzymes, hepatics); having influence on the kidney and the lower urinary tract (diuretics); having influence on the eye (ophtalmics); having influence on the skin (dermatotherapeutics); substances for the prophylaxis and therapy of infection diseases (pharmaceuticals with antibacterial influence, antimycotics, chemotherapeutics for virus and protozoal diseases, anthelmintics); having influence on malignant tumors (antimetabolites, cytostatics, topoisomerase inhibitors, mitosis inhibitors, antibiotics having cytostatic influence, hormones and hormone antagonists); having influence on the immune system and substances having immunological influence (serums, immunomodulators, immunosuppressives).

Plant protective agents are, for example, insecticides, herbicides, pesticides and fungicides.

Exemplified compounds and compound classes of synthetic agents are phenothiazines and analogues thereof, butyrophenones and diphenylbutylpiperidines, benzamides, benzodiazepines, hydroxytryptophans, caffeines, amphetamines, opioids and morphines, phetidines and methadones, derivatives of salicylic acid and acetylsalicylic acid, derivatives of arylpropanoic acid, derivatives of anthranilic acid, derivatives of aniline, derivatives of pyrazoles, sulfapyridines, hydroxychloroquine and chlororoquine, penicillamine, N-methylated barbiturates and thiobarbiturates, dipropylacetic acids, hydantoins, dopamines, noradrenaline and adrenaline, ergot alkaloids, derivatives of carbaminic acid, esters of phosphorous acid, belladonna alkaloids, hypothalamus hormones, HVL hormones, hypophysis hormones, thiouraciles and mercaptoimidazoles, sulfonylureas, histamines, triptanes, prostaglandins, dipyradimoles, hirudines and derivatives of hirudine, thiazides, psoralens, benzylperoxides and azelaic acid, vitamin A, vitamin K, vitamin B₁, B₂, B₆, nicotinic acid amide, biotin, vitamin B₁₂, vitamin C, halo compounds, aldehydes, alcohols, phenols, N-containing heterocycles, pyrethrins and pyrethroids, esters of phosphorous acid, esters of thiophosphorous acid, esters of carbaminic acid, β-lactams, aminoglycosides, tetracyclines, fluorochinolones, oxazolidinones, diaminobenzylpyrimidines, pyrazineamides, griseofulvine, aziridines, actinomycines, anthracyclines, cytokines, monoclonal and polyclonal antibodies. Further, antigen determinants, lectins, flavylium compounds, flavones and isoflavones as well as monosaccharides and oligosaccharides can be mentioned.

The synthetic agents can also be prepared by using natural agents. Further, said term comprises also potential agents as well as substances having pharmacophores as well as the frame (scaffold), said pharmacophores are attached to.

As already initially mentioned, in particular, the novel method for the selective separation of said substrate is suited to obtain information whether an arbitrary substrate can generally interact with a natural receptor. Conversely, it is also possible by use of, for example, all groups relevant for substrate recognition, to produce libraries of synthetic molecular regions, thus epitopes, whose parts each contain two, three, or also more different interaction sites. If, for example one contacts a known agent with said synthetic receptor libraries, a probability information is obtained about the type of the binding site at the natural receptor.

Thus, a new complementary principle is employed in the invention comprising on the side of the receptor respectively the sorbent and on the side of the substrate at least two different residues from compounds or groups, respectively, being responsible for the binding in compounds. Preferably, thereby, the compounds are selected from the group comprising amino acids, sugars, nucleotides, nucleosides, pyrimidine bases and purine bases.

However, from all possible combinations of said bivalently molecular regions among each other, only a small selection is compatibly complementary, that is energy-favored in its interaction. The multitude of the combinations is energy-unfavored, for example all pairs of hydrophobic residues on the one hand, and hydrophilic residues on the other hand, or all residues which repel each other.

For example, compatible are the combinations of pairwise groups capable of binding OH/phenyl with amino/alkyl residue, however not OH/phenyl with alkyl/amino residue, because only the hydrophilic OH and amino residues as well as the hydrophobic phenyl and alkyl residues bind each other. Further compatible combinations are, for example, carboxyl/amino with amino/carboxyl residue as well as imidazole/hydroxyl with amide/amide residue. Non-compatible in the meaning of said consideration is the combination hydroxyl/phenyl with alkyl/amino residue, because a hydrophilic residue cannot bind a hydrophobic residue.

With respect to twenty natural amino acids, for doublets of components having each at least one group capable of binding, all in all 380 variants will result. For a library including solely the meaningful structure variants, however, one needs essentially less of said synthetic doublets of components which can also be termed as doublet receptors, because in a series of amino acids the functionality is the same, such as for threonine and serine, for glutamine and asparagine, for valine, isoleucine and leucine, etc. Therefore, in general, it is sufficient to employ from said twenty amino acids preferably solely up to seven.

Since the moveably attached receptor groups in the synthetic receptor are able to change their space coordinates according to the requirement of the substrate, for the desired binding purpose frequently not the amino acids themselves with their differently long chains are needed, but only the principle that is needed for the interaction. In this meaning, often the functions of, for example, arginine, lycine, tryptophan and histidine are simply presentable by amino groups, provided only the function of the bases is needed.

If, for example, in the meaning of the invention, from seven amino acids solely four amino acids or the principle of said amino acids is used, simply 35 different combinations of doublet receptors will result after permutation.

Thus, another object of the invention is also a combinatorial library comprising a collection of sorbents having at least two different groups capable of binding at least one substrate having each at least two different groups, whereby the at least two different groups of the sorbents, respectively, and those of the at least one substrate are complementary towards each other.

Preferably, said combinatorial library is characterized thereby that the at least two different groups of the sorbents and the at least two different groups of the at least one substrate are selected among groups which are parts of different amino acids, sugars, nucleotides, nucleosides, pyrimidine bases or purine bases.

In another embodiment, the combinatorial library is characterized in that the manufacture of the sorbents comprises the steps (i) and (ii):
(i) determining at least two different groups capable of binding of a first synthetic or natural substrate to a sorbent,
(ii) applying at least two different groups capable of binding a second synthetic or natural substrate onto a carrier each thereby forming at least one sorbent, respectively, whereby the groups are groups that are the same groups of step (i) or are complementary to the groups of step (i), and the second substrate of step (ii) is the same substrate as the substrate according to step (ii) or is different from the first substrate according to step (i).

Another object of the invention is also a sorbent/substrate complex obtained in the selective separation of the substrate. Said sorbent/substrate complex comprises at least one sorbent with at least two different groups capable of binding and at least one substrate having at least two different groups capable of binding, whereby the groups capable of binding of the at least one sorbent and the groups of the at least one substrate are complementary towards each other.

Preferably, the at least two different groups of the at least one sorbent and the at least two different groups of the at least one substrate comprise different groups which are parts of amino acids, sugars, nucleotides, nucleosides, pyrimidine bases or purine bases.

In the sorbent/substrate complex, the binding between the at least one sorbent and the substrate exists in a non-covalent, covalent-reversible or covalent-irreversible bond. Preferably, the bond is non-covalently reversible.

Another object of the invention is also the use of the new method for the selectively binding of a substrate to sorbents by way of at least bivalent bonds and the use of the combinatorial library.

An application possibility is the detection of receptor/agent interactions as well as the agent screening.

Preferably, for the detection of receptor/agent interactions as well as for the agent screening, the above listed agents respectively classes of agents are employed.

Also for the development of new agent candidates (lead substances), the invention can be advantageously used. Said lead substances can be optimized with regard to their activity, selectivity, bioavailability, pharmacokinetics, and toxicity by using the new method respectively the combinatorial library.

Thereby, it is also conceivable that agent candidates interact only with one section of the biological binding site. By way of combination and connection of at least two of such agent candidates that bind at least two sections of the biological binding site, one simply can find new agents. Said agent search also works in using a highly paralleled method realization.

Another application possibility is the separation of stereoisomeric compounds and compounds with isomeric structures.

Further, the purification and/or separation of substrates and substrate mixtures is possible.

Preferably, the purification and/or separation is carried out by way of chromatographical methods. Electrophoresis, electrofocusing, gel electrophoresis, flat bed gel electrophoresis, parallel chromatography, parallel flash chromatography and capillary techniques can be mentioned as further suited methods. In case of sufficiently high selectivity, also a substrate can be directly adsorbed from the dissolved mixture by addition of the sorbent, can be stirred out and be isolated by filtering in form of a sorbent/substrate complex.

Further application possibilities are the removal of harmful substances and degradation products from substance mixtures, whereby the substances can also be present in very low concentration.

Preferably, harmful substances and degradation products can be separated off from body liquids, such as blood. For example, said harmful substances and degradation products exist in toxications, as metabolic products or metabolites. They can be of biogenous nature or can be formed in the body itself, however, they can be externally applied to said body, for example via the skin, via the oral mucosa or via injection, for example into the blood stream. Among harmful substances and degradation products are also snake venoms and intoxicants.

Preferably, the new sorbents can be applied in devices for dialysis.

Furthermore, the removal of harmful substances from solvents, from process waters and from processes for the manufacture of foodstuffs is possible.

By means of the invention, also pharmacokinetical tests can be carried out, particularly for the metabolisation and bioavailability.

The novel method for the selectively binding can also be advantageously used for the depletion of dynamically combinatorial libraries. For this, advantageously, from a mixture that contains besides a plurality of educts also desired substrate, preferably an agent, the latter is separated off according to the invention. Hereupon, in the mixture, the equilibrium is re-adjusted under formation of further substrate. The method of the separation is repeated as often until no further substrate is formed.

As discussed above, the novel methods are used for the targeted and selective separation of a substrate from a mixture with at least one further substrate.

Thus, according to the invention, the term selectively binding has the meaning that a substrate is separated off from a mixture with at least one further accompanying substrate thereby that the substrate having at least two different groups forms a stronger bond with the at least two different groups of the sorbent than the accompanying substrate.

With the present invention, for the first time, inter alia, the interaction site and the interaction type are exactly definable by way of the following methods, as it becomes apparent at hand of the examples:
by targetedly inserting binding sites at the receptor in the desired concentration and combination,
by omitting, adding, varying or blocking individual binding sites both at the receptor and also at the test substrates, whereby the effect for the binding strength is exactly (=energetically) determined, respectively,
by spectroscopically testing and by determining the adsorption isotherms.

For example, by way of comparison with the literature-known individual contributions of the respective non-covalent binding types, the overall interaction of a multivalent bond can be surprisingly well predicted. If the respective bond energy is determined in the expected amount, conversely, the conclusion to the groups that are involved in the bond is possible.

Thus, for the first time, selectivity can be targetedly created with respect to an arbitrarily selected separation problem.

With respect to the target compound (substrate) to be isolated, the novel teaching includes the construction of a purpose-directed non-covalently multivalent interaction which is sufficiently distinguished from the non-covalent interactions with the competing substrates (accompanying substances).

The methods of the present invention exhibit high values for the separation selectivity which also is simply termed as selectivity. Thereby, the separation selectivity a is defined as quotient of the respective bond constants respectively capacity factors of the bond of the substrate to be selectively separated to the sorbent, and the bond constant of the bond of the accompanying substrate to the sorbent.

For example, in omitting a single carboxyl group in a substrate, the separation selectivity reaches a value of more than 35. In exchanging an aromatic one ring system into a three ring system, a value of 10 is obtained.

With the novel method, in the technical scale separation selectivities can be achieved which, compared to the prior art, are surprisingly high, and which often allow separations which until now were not chromatographically possible.

Preferably, the separation selectivity a, by way of which the substrate to be selectively bound having the at least two groups capable of binding to at least one sorbent is separated off from a substrate mixture by way of using the at least one sorbent, is more than 1.4.

Preferably, the separation selectivity a is more than 2, more preferred more than 4, still more preferred more than 8.

More preferred are separation selectivities of more than 10, more preferred more than 35.

Furthermore, because the bond constant directly correlates with the determination of the Gibbs energy being known to the skilled person, also a correlation is given between Gibbs energy and separation selectivity. The more negative the change of the Gibbs energy ΔG is for the non-covalent bond, that is the stronger the complementary character of the groups binding each other is developed, also the higher is the separation selectivity towards accompanying substances which, by way of inserting the groups capable of binding (with the target substances), do not noteworthily change with respect to the Gibbs energies (to said groups respectively to the sorbent).

Moreover, for creating selectivity with respect to an arbitrary substrate pair, it is sufficient to additionally attach one group capable of binding in the sorbent, as far as said group does not have a complementary partner for one of both substrates to be separated.

In the described manner, it can also be detected, whether and which bond types simultaneously exist (i.e. multi valence). Said multi valence, in particular the achieved set values for the bond constants and for the Gibbs energy are, however, only then possible, if the substrate can be at least partially spatially embedded by induced fit or conformatively adapts itself to the receptor.

Said adaptation is preferably possible with the polymeric network, whereby the cross-linking degree of the polymeric nano film is selected in a way that kill sufficiently conformative movability and therewith adaptation capability to the substrate structure is given. Preferably, small substrates with molar masses below 1000 Da are completely embedded within the polymeric network. Preferably, larger substrates, such as peptides or proteins, bind with limited contact area in a deepening in the polymer net which allows a multivalent interaction, however, avoids by inclusion a binding being too strong.

In order to realize the concept for the construction of selective multivalent binding sites, it is frequently necessary to offer the required binding sites conformatively movable in the space. Moreover, it is necessary to offer a sufficiently strong binding tendency by the substrate in order to achieve the conformative adaptation (induced fit). Last but not least, the at least two necessary interaction sites must be pre-organized in the space in a high concentration in order to realize the desired binding event in a large number on the basis of the conformation change.

### BRIEF DESCRIPTION OF THE DRAWINGS

FIG. 1. Structure of Naringenine.

FIG. 2. Scatchard diagram for different substrate concentrations [S] of 4-amino-3-nitrobenzonitrile.

The invention is illustrated by the following examples.

### EXAMPLE 1

### Selectively Binding of N-Blocked Amino Acids as Substrates to Sorbents on Basis Polyvinyl Amine/Silica Gel by Way of at Least Bivalent Bonds

The retention properties of eight different derivatives of amino acids (substrates in Table 1) were tested at four different sorbents based on polyvinyl amine/silica gel (sorbents in Table 2), whereby as test method the chromatography was chosen. In the following, the sorbents are also termed as stationary phases, synthetic receptors are also termed as receptors, also in the other Examples.

The amino acid derivatives were derivatives of glutamine (1-4) and glutamate (5-8), whose amino groups were blocked with the four different protective groups acetyl (Ac), tert.-butyloxycarbonyl (Boc), benzyloxycarbonyl (Z) and fluorenyl-methoxycarbonyl (Fmoc), respectively.

TABLE 1
Employed substrates

| glutamine de- | | glutamate de- | | | |
|---|---|---|---|---|---|
| rivatives | | rivatives | | | |
| 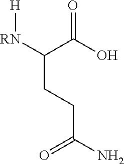 | | 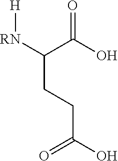 | | N-protective group | |
| Ac-Gln | 1 | Ac-Glu | 5 | Ac | 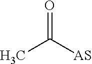 |
| Boc-Gln | 2 | Boc-Glu | 6 | Boc | 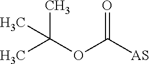 |
| Z-Gln | 3 | Z-Glu | 7 | Z | 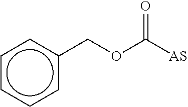 |
| Fmoc-Gln | 4 | Fmoc-Glu | 8 | Fmoc | 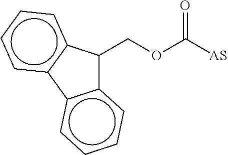 |

The used receptor phases were polyvinyl amine-coated spherical silica gel with a particle size of 20 μm and a pore diameter of 1000 Å. During the coating method, at first the amino phase A was produced. The derivatized receptor phases B to D were synthesized from the amino phase A by means of solid phase synthesis according to known methods.

TABLE 2
Structure of the employed receptor phases

| phase name | | phase composition | phase structure |
|---|---|---|---|
| A | BV 02043 | K1000-PVA-FA-2-5-Dod amino phase | 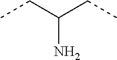 |
| B | ND 03001#2 | K1000-PVA-FA-2-5-Dod-Ac-100 acetyl phase | 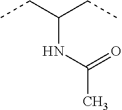 |
| C | ND 02031 | K1000-PVA-FA-2-10-Dod-MVS- 100 4-methylvaleryl phase | 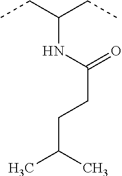 |
| D | ND 03017 | K1000-PVA-FA-2-5-Dod-BzlO- 100 benzyloxycarbonyl phase | 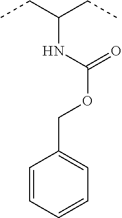 |

All receptor phases still had a measurable content of free amino groups which could undergo ionic interactions in the protonated state with appropriate anionic groups of the substrate, for example carboxylate groups. Additionally, the receptors C and D contained a residue suitable for lipophilic interactions.

The amino group content of the receptors was determined from chromatographical breakthrough curves with 10 mM 4-toluenesulfonic acid in DMF. The determined quantities of amino groups per gram receptor phase are summarized in Table 3.

TABLE 3
Amino group content of the receptor phases

| synthetic | amino groups |
|---|---|
| receptor | in mmole/g |
| A BV 02043 | 0.60 |
| B ND 03001#2 | 0.03 |
| C ND 02031 | 0.13 |
| D ND 03017 | 0.16 |

For the chromatographical tests at the substrates 1 to 8, aqueous tris-HCl-buffer having pH 7.5 was used as mobile phase. The elution was carried out under isocratic conditions with buffer concentrations of from 10 to 500 mM.

As measure for the strength of the interaction between substrate and receptor in the respective buffer solutions, the device-independent relative elution factor k′ (capacity factor) was used. It can be calculated from the difference of elution volume at the maximum peak and the column dead volume divided by the column dead volume, as illustrated in the following equation:

$${k}^{′} = \frac{elution volume - column dead volume}{column dead volume}$$

The k′-values of the substrates in 10 mmolar respectively 50 mmolar tris-HCl-buffer are summarized in the Tables 4 and 5.

TABLE 4
k′-values of the substrates in 10 mmolar tris-HCl-buffer (pH 7.5)

| | relative elution value k′ of the substrates | | | | | | | |
|---|---|---|---|---|---|---|---|---|
| | Ac | | Boc | | Z | | Fmoc | |
| receptor | 1 | 5 | 2 | 6 | 3 | 7 | 4 | 8 |
| A | 9.5 | 433 | 8.8 | 411 | 15 | >715 | 85 | >715 |
| B | 0.1 | 0.2 | 0.1 | 0.3 | 0.3 | 0.3 | 0.2 | 1.6 |
| C | 1.3 | 25.7 | 3.6 | 127 | 12.5 | 575 | 419 | >715 |
| D | 1.4 | 26.1 | 2.1 | 41.9 | 9.7 | 263 | 297 | >715 |

TABLE 5
k′-values of the substrates in 50 mmolar tris-HCl-buffer (pH 7.5)

| | relative elution value k′ of the substrates | | | | | | | |
|---|---|---|---|---|---|---|---|---|
| | Ac | | Boc | | Z | | Fmoc | |
| receptor | 1 | 5 | 2 | 6 | 3 | 7 | 4 | 8 |
| A | 2.3 | 33.2 | 2.2 | 34.4 | 3.6 | 62.9 | 20.3 | 478 |
| B | 0 | 0 | 0 | 0 | 0 | 0.1 | 0.2 | 0.2 |
| C | 0.2 | 2.3 | 0.9 | 9.9 | 3.4 | 37.6 | 111 | 562 |
| D | 0.3 | 2.6 | 0.5 | 5.0 | 2.5 | 20 | 78.4 | >715 |

The comparison of the k′-values within and between the Tables 4 and 5 provided the following observations and interpretations of the observations:

1. Observation: The acetylated receptor phase B (ND 03001#2) did bind even in the lowest buffer concentration none of the substrates in a noteworthy quantity (k′≦1.6).

Interpretation of the observation: Said receptor contains only very few amino groups for possible ionic interactions with the carboxylate groups of the substrates. Neither the acetyl groups nor the polyvinylamine chains of the receptor phase are capable of undergoing important lipophilic interactions.

2. Observation: In 10 mmolar buffer, the substrates with two carboxylate groups (5-8) did bind with a factor of approximately 20 to 40 stronger than the corresponding substrates with only one carboxylate group (1-4). In 50 mmolar, the binding of the dicarboxylates was still stronger with a factor of 10 to 25 than the binding of the monocarboxylates.
Interpretation of the observation: Obviously, there are ionic interactions between carboxylate groups of the substrates and the amino groups of the receptors. Based on the bivalence of the interaction, for dicarboxylates, said interactions result in a much more stronger bond than for substrates with only one carboxyl group. In aqueous medium, the amide group does not contribute a noteworthy bond contribution.
3. Observation: In increasing the buffer concentration from 10 to 50 mmolar, the binding strengths decreased, for monocarboxylates for a factor of approximately four, for dicarboxylates for a factor of approximately ten.

Interpretation of the observation: Also this result can be explained from ionic interactions that are weakened for higher buffer concentration. Obviously, the weakening results from the competition of the buffer salts with the carboxylate groups of the substrates for the ammonium groups of the receptor. In case of the strong binding of the substrates 5-8, the competition of the buffer salts has a stronger effect because two carboxylate groups thereof are affected.

4. Observation: For elsewise identical substrates, the k′-values drastically increased with the size of the organic residue of the N-protective group. The magnitude of said binding increase was independent from the buffer concentration.

Interpretation of the observation: Therewith, it is shown that besides the ionic interactions between the carboxylate groups of the substrates and the ammonium groups of the receptors additionally lipophilic interactions are present between substrate and receptor. Thus, in the transition from small to large organic residues in the N-protective group, in particular, the binding strengthening has an effect on receptor phases C and D, whose receptor groups are particularly suited for lipophilic interactions.

Conclusion: With the experiments described above, it clearly could be verified that the synthetic receptors simultaneously can undergo two or three binding interactions with appropriate substrates, provided receptor and substrate are complementary with respect to their functional groups.

Hence, it can be concluded that by design of a receptor that is appropriately complementary to a target substance, accompanying substances or by-products can be easily separated off. The measure for the realization of the separation is the quotient from the k′-values, the selectivity alpha that is specified in the following formula:

Selectivity: alpha=k₂′/k₁′

For example, alpha was approximately 25 (263/9.7) with the benzyl/amino receptor phase D for the chromatographical separation of Z-Gln (3) and Z-Glu (7) with 10 mmolar tris-HCl-buffer (pH 7.5) as mobile phase.

Therewith, it could be shown that a quantifiable correlation existed between the respective targetedly inserted molecule residues and the binding strengths.

### EXAMPLE 2

### Binding of the Flavanone Naringenine as Substrate to Receptor Phases of the Company InstrAction by Way of at Least Bivalent Bonds

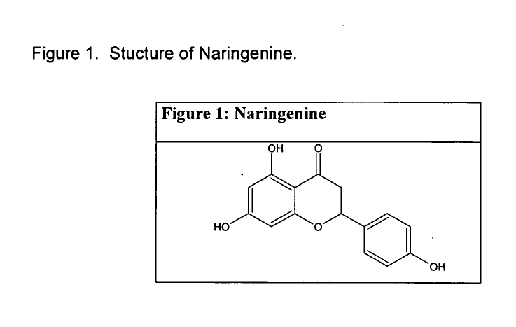

The interaction between naringenine (FIG. 1) and seven different receptor phases of the company instrAction (Tables 6 and 7) was measured in acetonitrile as solvent. For said measurements, the direct method of the equilibrium determination was used in the so-called “stirred beaker experiment”

TABLE 6
Employed stationary phases

| receptor | | amino groups |
|---|---|---|
| phase | phase composition | in mmole/g |
| A BV 02051 | K1000-PVA-FA-2-5-Dod | 0.54 |
| C ND 02048#2 | K1000-PVA-FA-2-5-Dod-MVS-100 | 0.16 |
| D ND 03017#3 | K1000-PVA-FA-2-5-Dod-BzlO-100 | 0.10 |
| E ND 03033#2 | K1000-PVA-FA-2-5-Dod-ImAc-100 | 0.53 |
| F ND 03049 | K1000-PVA-FA-2-5-Dod-Acrid9Car- | 0.29 |
| | 100 | |
| G ND 03050 | K1000-PVA-FA-2-5-Dod-NaphCar-100 | 0.23 |
| H ND 03062 | K1000-PVA-FA-2-5-Dod-iNic-100 | 0.35 |

For the stirred beaker experiments, exactly weight quantities of receptor phase (each approximately 100-300 mg) were suspended in exactly measured volumes of solvent (15 ml). To these suspensions, in portions, exactly measured quantities of naringenine were added (for example 1.0 ml of a 10 mmolar solution in acetonitrile). The naringenine partitioned between the receptor phase and the solvent in establishing a dynamic equilibrium.

The state of equilibrium could be exactly determined by determination of the naringenine concentration in the solvent via high performance liquid chromatography (HPLC). From this, one directly obtained the substance quantity of the naringenine in the liquid phase (acetonitrile). The substance quantity of the naringenine in the receptor phase was calculated as difference between added naringenine and naringenine in solution. In each stirred beaker experiment, the equilibrium was repeatedly determined (6-12 times) with increasing naringenine concentration in the system. For the resulting naringenine and solvent additions as well as removals, the balance was carefully made up and taken into account for the calculation of the substance quantities.

TABLE 7
Derivatives of the stationary phase

| name | abbr. | structure |
|---|---|---|
| 4-methylvaleric acid group | MVS | 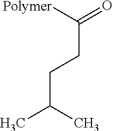 |
| benzyloxycarbonyl group | BzlO | 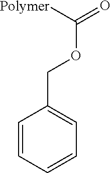 |
| 4-imidazolylacetic acid group | ImAc | 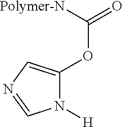 |
| acridine-9-carboxylic acid group | Acrid9Car | 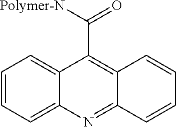 |
| 2-naphthylcarboxylic acid group | Naphcar | 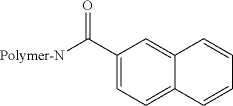 |
| isonicotinic acid group | iNic | 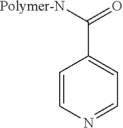 |

For each equilibrium establishment, one obtained one point on the adsorption isotherm (plot of receptor-bonded naringenine [RS] versus naringenine in solution [S]). By using the Langmuir model for the adsorption isotherms, the equilibrium constants for the association (K_{A}) and the maximum chargebility [R₀] were calculated by non-linear regession.

Langmuir isotherm: [RS]=[R₀]×[S]/(1/K_{A}+[S])

For particularly weak interactions, the method of the non-linear regression failed. In said circumstances, K_{A} and R₀ were determined by linear regression from the diagram according to Scatchard ([RS]/[S] plotted versus [RS]).

In the plot according to Scatchard, simple Langmuir isotherms are straight lines:

Scatchard linearization: [RS]/[S]=−K_{A}×[RS]+K_{A}×[R₀]

An important advantage of the Scatchard plot is that deviations from the linearity can be easily detected. Such deviations can indicate receptor phases simultaneously having binding sites of different binding strengths and bond numbers.

The values for the association constant K_{A} and the maximum chargebility R₀ are presented in Table 8:

TABLE 8
Association constant K_{A} and maximum chargebility R₀

| | | interaction | | | |
|---|---|---|---|---|---|
| receptor | | strong | | weak | |
| phase | derivative | K_{A2} | R₀₂ | K_{A1} | R₀₁ |
| A BV 02051 | 100% amino groups | 2121 | 14.7 | 931 | 22.1 |
| C ND 02048#2 | MVS-100 | 1302 | 48.4 | 760 | 66.3 |
| D ND 03017#3 | BzlO-100 | — | — | 329 | 29.2 |
| E ND 03033#2 | ImAc-100 | 6194 | 4.9 | — | — |
| F ND 03049 | Acrid9Car-100 | 2961 | 19.5 | 65 | 243 |
| G ND 03050 | NaphCar-100 | 1943 | 38.5 | 379 | 91 |
| H ND 03062 | iNic-100 | — | — | 778 | 21.7 |

Observations and interpretation of the observations: Based on its phenolic hydroxyl groups, naringenine could form polar interactions with the primary amino groups of the amino phase A. In the aprotic solvent acetonitrile, said interactions could be well measured. The existence of strong (K_{A2}) and weak binding sites (K_{A1}) can be interpreted in a manner that naringenine obviously has the possibility to form monovalent, bivalent and trivalent polar bonds, corresponding to the three existing phenol groups.

In the receptor phases C, D and G, most of the primary amino groups of phase A are derivatized with lipophilic residues. If said residues would not contribute to the binding of the naringenine, the chargebilities R₀ of said phases would have to decrease corresponding to the lower amino group content. The equilibrium constants should approximately remain the same because the type of the interaction would still not change. As a matter of fact, the chargebilities partially clearly increased, for example from 14.7 to 38.5 mmole/g phase for the naphthoyl-derivatized receptor (receptor phases A and G). Said result can only be explained with additional interactions between naringenine and the derivatization groups. Said free receptor groups have in common to be able to undergo lipophilic interactions. On its part, naringenine has also lipophilic molecule portions in order to share such interactions.

From this follows that naringenine could simultaneously realize polar bonds with the receptor phases C, D and G, i.e. with the still remaining amino groups, and lipophilic bonds with the receptor groups MVS, BzlO respectively NaphCar. The circumstance is remarkable that said lipophilic bonds could be observed in an organic solvent (acetonitrile). That means that between naringenine and the lipophilic receptor groups contacts take place which compete in energy with a solvation of the lipophilic group with an organic solvent.

Therefore, the association constants K_{A} with the receptor phases C, D and G are composed from contributions of polar and lipophilic bonds. Throughout, the association constants are lower here than with the amino phase A. Obviously, the lipophilic bonds are weaker than the polar bonds, what in turn can be attributed to the employed relatively polar organic solvent (acetonitrile).

The receptor phases E, F and H contain receptor groups which can both take part in lipophilic and polar bonds—all three contain amino groups being embedded in partially extended aromatic structures. Indeed, both the highest K_{A}-values can be found with the receptor phases E and F. It can be presumed that here an cooperative coaction of the polar and the lipophilic bond contributions were particularly favored, whereas in the receptors C, D and G lipophilic receptor groups were incorporated at the cost of amino groups.

Result: In this Example, it was shown that in one solvent a substrate (naringenine) can have different bonds towards appropriate receptor phases. In suited choice of the receptor groups in the stationary phase, polar and lipophilic interactions for the binding of the substrate can be simultaneously activated. Accordingly, receptor phases can be synthesized which are optimized for the binding of particular substrates or substrate groups, because different binding possibilities are simultaneously present and thereby selective interaction spaces are created.

### EXAMPLE 3

### Binding of Structurally Related Benzene Derivatives as Substrates to a Receptor Phase of the Company InstrAction by Way of at Least Bivalent Bonds

The interaction between structurally related benzene derivatives and an instrAction receptor phase C(ND 02048#2, K1000-PVA-FA-2-4-Dod-MVS-100) was measured in a non-polar organic solvent mixture. Besides the 4-methylvaleric acid groups (MVS), the receptor phase C also contained 0.16 mmole/g amino groups. The solvent was a mixture of methyl-t-butyl ether/heptane (1 part/3 parts by volume). In said non-polar solvent mixture, on one hand, predominantly polar inter reactions were to be expected, and on the other hand, all substances to be tested were well soluble therein.

The association constants (K_{A}) and the maximum chargebility (R₀) for the interaction between the receptor phase and the test substances were determined in so-called “stirred beaker experiments”.

For the stirred beaker experiments, exactly weight quantities of receptor phase (each approximately 200-350 mg) were suspended in exactly measured volumes of solvent (15 ml). To said suspension, exactly measured substrate quantities were added in portions. The substrate to be tested partitioned between the receptor phase and the solvent in establishing a dynamic equilibrium. The state of equilibrium could be exactly determined by determining the substrate concentration in the solvent via high performance liquid chromatography (HPLC). Here, one directly obtained the substance quantity of the substrate in the solvent. The substrate quantity of the substrate at the receptor phase was calculated as difference between added substrate and substrate in solution. For each stirred beaker experiment, the equilibrium was repeatedly determined (6-12 times) with increasing substrate concentration in the system. The balance was carefully made up for the substrate and solvent additions and removals and taken into account for the calculation of the substance quantities.

For each establishment of the equilibrium, one obtained one point on the adsorption isotherm (plot of receptor-bound substrate [RS] versus substrate in solution [S]). By using the Langmuir model for the adsorption isotherm, the equilibrium constant for the association (K_{A}) and the maximum chargebility (R₀) was calculated by non-linear regression:

Langmuir isotherm: [RS]=[R₀]×[S]/(1/K_{A}+[S])

The method of the non-linear regression failed for particular weak interactions. In said cases, K_{A} and R₀ were determined by linear regression from the diagram according to Scatchard ([RS]/[S] plotted versus [RS]). In the plot according to Scatchard, the simple Langmuir isotherms are straight lines:

Scatchard linearization: [RS]/[S]=−K_{A}×[RS]+K_{A}×[R₀]

In Table 9, the obtained interaction parameters K_{A} and R₀ are presented together with the test substrates:

TABLE 9
Employed test substances and binding results

| | substrate | K_{A} in l/mole |
|---|---|---|
| substrate name | structure | R₀ in μmole/g phase |
| 4-amino-3-nitrobenzonitrile | 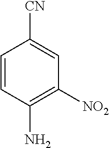 | 17,700 ± 2,600 3.5 ± 0.3 |
| 3-nitrobenzonitrile | 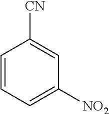 | 405 ± 109 12.9 ± 2.4 |
| 4-aminobenzonitrile | 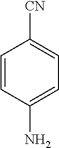 | 991 ± 59 19.6 ± 0.7 |
| benzonitrile | 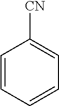 | 27 ± 14 not exactly measurable |
| nitrobenzene | 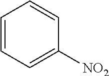 | immeasurably small in the used system |

Observations of the results: One can see from Table 9 that the strength of the interaction between test substance and receptor phase that is represented by the association constant K_{A}, increased with the number of the substituents at the benzene ring.

Benzene rings with only one substituent had association constants of below 40 l/mole, values being at the border of the measurability in the described measuring system.

A second substituent at the benzene ring contributed a further interaction possibility to the test molecule. Both weak interactions cooperated and yielded association constants for the substituted benzene derivatives which approximately presented the product of the association constants of the monosubstituted benzenes. Accordingly, K_{A}-values of from 400 to 1,000 l/mole were obtained.

The third substituent at the benzene ring multiplied the association constant of the disubstituted benzene with its own, relatively weak interaction potential (K_{A}˜20-40 l/mole), and one obtained an association constant of 17,722 l/mole for the benzene with the three substituents.

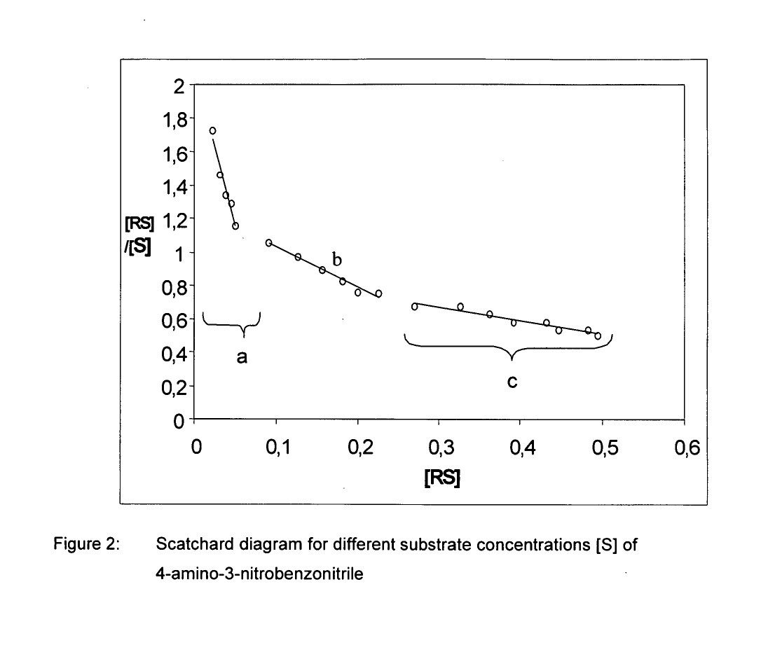

In FIG. 2, the Scatchard diagram is presented for 4-amino-3-nitrobenzonitrile. Therein, a, b, and c have the following meaning:
a: region of trivalent interactions
   [S]=0.0044-0.043 mmole
   K_{A3}=17,722 l/mole
   R₀₃=3.5 μmole/g phase
b: transition region of trivalent and bivalent bonds
   [S]=0.086-0.30 mmole
   K_{A2}=2,350 I/mole
   R₀₂=16 mole/g phase
c: region of bivalent interactions
   [S]=0.40-0.98 mmole
   K_{A1}=855 l/mole
   R₀₁=33 μmole/g phase

From the Langmuir isotherm, one did not only obtain the strength of the interaction in form of the association constant K_{A}, however, also the number of the interaction sites as maximum chargebility R₀. The maximum chargebility for the trivalent interaction was approximately five times lower than R₀ for the bivalent interaction. This is directly understandable because one can presume that in the synthetic receptor phase fewer binding sites for three simultaneous interactions are present compared to two or even only one interaction. Additionally to the trivalent binding sites, 4-amino-3-nitrobenzonitrile could also occupy bivalent and even monovalent binding sites; naturally with appropriately lower binding strengths (K_{A}) and higher maximum chargebilities (R₀).

Said circumstance is illustrated in FIG. 2. If one determined the parameters K_{A} and R₀ with very low substrate concentrations, then one predominantly observed the strong, trivalent interaction (K_{A3} and R₃). The weaker monovalent and bivalent binding sites were not noteworthily occupied from such diluted solutions. If one determined K_{A} and R₀ with higher substrate concentrations, one obtained the interaction values of the weaker and more numerous bivalent binding sites (for example K_{A1} and R₀₁). For these substrate concentrations, strong binding sites were already saturated and provided only a constant contribution to the adsorption isotherm. Monovalent interactions are not illustrated in FIG. 2.

In general, in the Scatchard diagram, a curved course of the isotherm increasing to the left, proves the simultaneous presence of differently strong binding sites.

Result: With the presented experimental results it could be shown that the receptor phase C (structure K1000-PVA-FA-2-5-Dod-MVS-100) can undergo both strong trivalent and also weaker, monovalent and bivalent interactions with 3-amino-4-nitrobenzonitrile.

Towards substrates with a lower number of substituents, the same receptor phase behaves accordingly, that is the maximum binding strength complied with the number of substituents at the substrate molecule.

Moreover, the strength of the bond could be influenced by the substituent-dependent change of the permanent and induced dipoles of the substrate molecule.

### EXAMPLE 4

### Binding of Steroids as Substrates to Receptor Phases of the Company InstrAction by Way of at Least Bivalent Bonds

The binding (retention) of estradiol and of testosterone to a receptor phase A (SBV 01044 VD/4 in column PV 02067) that solely contained amino groups, and to a phase C(ND 02001/1 in column PV 02001) which was derivatized with branched alkyl groups (4-methylvaleric acid) in a degree of 27%, was determined by means of gradient HPLC.

For the gradient HPLC, the following conditions were used:
Neutral eluents:
Eluent A: 1 part dimethylformamide+9 parts water (parts per volume)
Eluent B: dimethylformamide
Acidic eluents:
Eluent A: 10 mmole trifluoroacetic acid (TFA) in 1 part dimethylformamide+9 parts water (parts per volume)
Eluent B: 10 mmole trifluoroacetic acid in dimethylformamide
Gradient profile: Constant eluent A with a flow rate of 0.2 ml/min for five minutes; then admixing of B with 2%/min at 0.6 ml/min until the complete substance elution.

In the gradient, the respective substance will elute if the Gibbs energy for the solvent method in the mobile phase just exceeds the receptor/substrate bond energy. Also, the Gibbs energy ΔG of the receptor/solvent interaction affects the energy balance: as a rule, the entropy ΔS is decreased because of the higher number of adsorbed smaller solvent molecules, and the interaction enthalpy ΔH is moderately negative.

For an appropriately composed receptor phase, during the substrate binding (adsorption) the interaction enthalpy ΔH of the solvent adsorption is considerably less negative than the contribution of the multivalent interaction enthalpy ΔH between receptor and substrate.

Because the examined substances were poorly soluble in water and well soluble in DMF, the DMF content of the mobile phase being necessary for elution was a rough measure which, however, could simply be determined in order to quickly compare the binding strength of several substrates towards a receptor.

It was expected that both estradiol and testosterone can undergo lipophilic interactions with the receptor phases, further, estradiol should be capable of an ion-like phenol/amine bond. Furthermore, the 4-methylvaleric acid group existing in receptor phase C(ND 02001/1 in column PV 02001) should considerably strengthen the lipophilic bond portion compared to amino phase A.

It was forecasted that, contrary to testosterone, estradiol can undergo a bivalent bond with a ionic and a lipophilic portion. In this case, estradiol should elute considerably later than testosterone from the receptor phase C in the used solvent gradient. For phase A, on the other hand, all in all clearly shorter retention times were to be expected as well as lower differences in the elution behavior of testosterone and estradiol.

In Table 10, the DMF content of the mobile phase is indicated which was required in order to break the receptor/substrate bond.

TABLE 10
Gradient elution of estradiol and testosterone

| | | | 10 mmole TFA water/DMF- | |
|---|---|---|---|---|
| | water/DMF gradient | | gradient | |
| | amino | receptor | amino | receptor |
| | phase A | Phase C | phase A | phase C |
| substrate | PV 02007 | PV 02001 | PV 02007 | PV 02001 |
| estradiol | 13.1% | 46.7% | 11.3% | 36.6% |
| testosterone | 10.0% | 18.5% | 10.0% | 27.3% |

1. Observation: The results indicated that estradiol bound stronger than testosterone already on the amino phase A (PV02007), what could be attributed to the additional ionic interaction. On the alkylated phase C (PV 02001) estradiol eluted not until at a concentration of 47.7% DMF, what, compared to the basic phase represented an increase of 33.6 parts per volume. With respect to the elution force of DMF, said result corresponded to a drastical increase in the binding. On the other hand, the binding of testosterone moderately increased to 18.5% DMF.
2. Observation: As could be expected, the binding of estradiol decreased if its ionic interaction possibility was largely eliminated by protonating the amino group at the stationary phase while adding 10 mmole trifluoroacetic acid to the mobile phase.

On the other hand, the binding of testosterone was moderately strengthened in the acidic medium with respect to the phase C, and remained unchanged with respect to the phase A. For both substrates, it was conceivable that the amino groups of the receptors which were created in the eluent because of the trifluoroacetic acid, undergoes additional interactions which are not available for the amine.

All in all, it was striking that the binding strengthening at the receptor phase was considerably higher if two different non-covalent interaction types were used. The binding strengthening by means of solely enlarging the lipophilic contact region of the aliphatic molecule parts was lowerly developed.

Furthermore, said results were supported by comparison of the retention of characteristic structure elements of the estradiol molecule. With such molecular probes, comprehensive HPLC tests could be fastly carried out. So, 2-naphthol did bind to phases of type C considerably stronger than naphthalene, and, in turn naphthalene better than 1,2,3,4-tetrahydronaphthol. In turn, the expected ionic binding contribution could be derived from said behavior, whereas a polar binding of the alcoholic OH groups expectedly did not occur in the aqueous solvent.

Result: The bivalent binding of phenolic steroids on phases containing alkyl and amino groups, such as C (PV02001), was advantageous for the separation from non-aromatic steroids. Thereby, under isocratic separation conditions, a values (separation selectivities) up to 10 were achieved.

On the other hand, on the weakly hydrophobic ion exchanger A (PV 02007), said separation was not possible with satisfying resolution.

The illustrated principle can be generalized for the separation of phenolic substances from neutral or basic aliphatics, however, also for aromatics. Furthermore, also multivalent phenols could be well separated.

### EXAMPLE 5

### Binding of Lactams as Substrates to Receptor Phases of the Company InstrAction by Way of at Least Bivalent Bonds

The binding of methylphenylhydantoin (MPH) 1, diphenylhydantoin (DPN) 2 and methylphenylsuccinimide 3 out from chloroform to a series of receptor phases containing 80% amino groups and 14% benzyl groups (for example PV 99047, PV 00010; cross-linking degree 5%) was determined by means of front analysis.

For this, the receptor phase which was packed in HPLC columns (40×4 mm) was rinsed with substrate solutions of increasing concentration until the respective saturation equilibrium. From the flow rate, from the time until the breakthrough of the substance and the substrate concentration, the respective concentrations of bound substrate [RS] can be calculated for the known constant substrate concentrations [S]=[S₀]. From the breakthrough curves which were measured for 10-12 substrate concentrations, via the adsorption isotherms respectively the Scatchard diagrams, the bond constants K_{A} and the saturation concentration [R_{O}] could be determined, whereby the regions of bivalent and monovalent bonds could be detected.

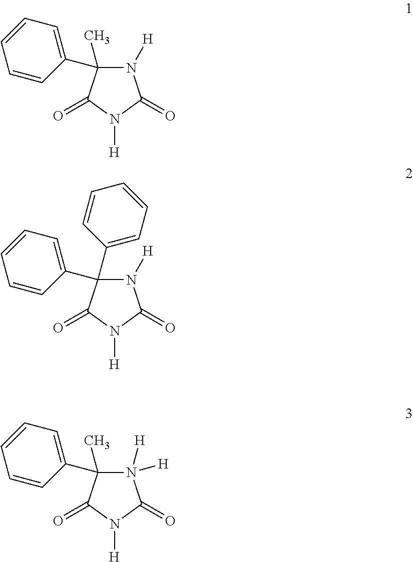

By means of the solvent selection, it was ensured that essentially polar interactions are realized, in particular hydrogen bonds.

Typical measured values are indicated under a) to c).
a) Binding of MPH to poly(benzyl-N-allyl-carbamate) on silica gel, 6 layers, cross-linked (PAA-OBz114-2Dod, PV 99047):
   Region of bivalent bonds:
   K_{A}=12,703 M⁻¹
   ΔG=5.50 kcal/mole
   R₀=12.0 μmole/g
   Region of monovalent bonds:
   K_{A}=221 M⁻¹
   ΔG=3.14 kcal/mole
   R₀=301.4 μmole/g
b) Binding of DPH to poly(benzyl-N-allyl-carbamate) on silica gel, 6 layers, cross-linked (PAA-OBz114-2Dod, PV 00010):
   Region of bivalent bonds:
   K_{A}=19,880 M⁻¹
   ΔG=5.76 kcal/mole
   R₀=4.6 μmole/g
   Region of monovalent bonds:
   K_{A}=201 M⁻¹
   ΔG=3.09 kcal/mole
   R₀=226.7 μmole/g
c) Binding of MPS to poly(benzyl-N-allyl-carbamate) on silica gel, 3 layers, cross-linked (PAA-OBz114-2Dod, PV 99047):
   Region of monovalent bonds:
   K_{A}=75-78 M⁻¹
   R₀=96.5-97.4 μmole/g
      1. Observation: For both hydantoins 1 and 2 (MPH and DPH), in comprehensive test series, bivalent bond constants K_{A} were determined between 6,000 and 23,000 M⁻¹ for saturation substance quantities R₀ between 3 μmole/g phase and 12.6 μmole/g phase for several variants of the receptor phases (for example PV 99047, PV 00010). This indicated that two hydrogen bridges could be formed towards the amine. The monovalent bond constant was between 109 and 221 M⁻¹ (R₀=239-301 μmole/g). On the other hand, for the succinic imide derivative solely monovalent bond constants of from 75 to 78 M⁻¹ were found with a saturation value R₀ of 96 μmole/g. This can be interpreted therewith that a succinic imide can only form one hydrogen bridge, and, therefore, is only capable of monovalently binding.
      2. Observation: The bond constant for a bivalent bond corresponds quite well with the product of the values of the combined monovalent bond constants. The corresponding monovalent Gibbs energies ΔG approximately add each other. For a single hydrogen bond of a lactam group of a five-membered ring, in chloroform Gibbs energies ΔG between 2.5 and 3.14 kcal/mole were determined at 25° C., and for the bivalent hydrogen bonds between 5.06 to 5.88 kcal/mole. These values exceed the data which were expected for the solvent chloroform at hand of the literature (MPH: K_{A}=6,014 M⁻¹, ΔG=5.06 kcal/mole, R₀=3.2 μmole/g; DPH: K_{A}=7,171 ΔG=5.16 kcal/mole, R₀=6.9 μmole/g and K_{A}=145 M⁻¹, ΔG=2.90 kcal/mole, R₀=264.0 μmole/g).

Result: Therewith, it could be shown that a bivalent binding strengthening also occurs then if on the substrate side and on the receptor side two similar complementary residues (binding site residues), respectively, interact with each other, similar to chelate effects. In the mentioned case, these were the amide groups of the substrates and the amine groups of the receptor. Thereby, the Gibbs energies approximately added each other, and the binding constants multiplied each other. According to said principle, in particular, receptor phases can be developed which are suited for the separation of homologous substances or of substances with different valence with respect to the functional groups (for example monohydric to hexahydric alcohols, such as sugars).

### EXAMPLE 6

### Binding of Some C-Blocked Amino Acids as Substrates to Sorbents on Basis Polyvinyl Amine/Silica Gel As Sorbents By Way of at Least Bivalent Bonds

The retention properties of 18 different amino acid derivatives (substrates in Table 11) were investigated in the chromatography on seven different stationary phases (synthetic receptors).

The amino acid derivatives (1-18) were esters of alanine, leucine, proline, lysine, histidine, phenylyalanine, tyrosine and tryptophan. The esters were selected in order to exclude undesired interactions of the ionizable carboxylate functions. We did not expect noteworthy interaction contributions from the methyl esters, very contrarily to the benzyl esters.

TABLE 11
Amino acid derivatives as substrates

| substrate | name | structure |
|---|---|---|
| 1 | H-Ala-OMe | 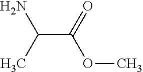 |
| 2 | H-Ala-OBzl | 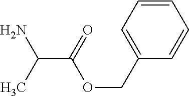 |
| 3 | H-Leu-OMe | 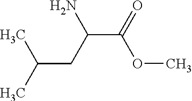 |
| 4 | H-Leu-OBzl | 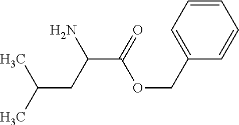 |
| 5 | H-Pro-OMe | 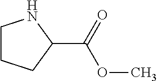 |
| 6 | H-Pro-OBzl | 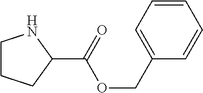 |
| 7 | Z-Lys-OMe | 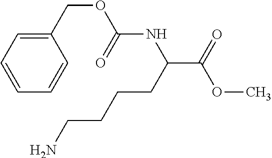 |
| 8 | H-Lys(Z)-OMe | 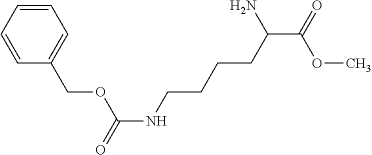 |
| 9 | Boc-Lys-OMe | 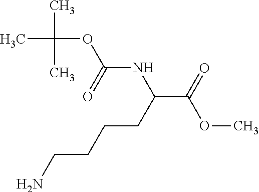 |
| 10 | H-Lys(Boc)- OMe | 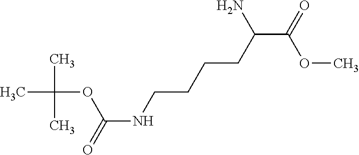 |
| 11 | H-His-OMe | 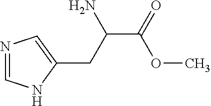 |
| 12 | Bzl-His-OMe | 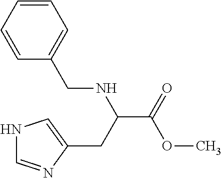 |
| 13 | H-Phe-OMe | 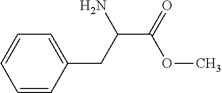 |
| 14 | H-Phe-OBzl | 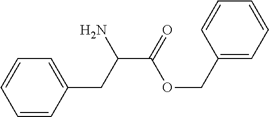 |
| 15 | H-Tyr-OMe | 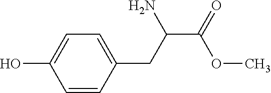 |
| 16 | H-Tyr-OBzl | 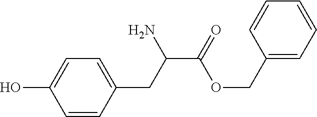 |
| 17 | H-Trp-OMe | 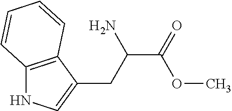 |
| 18 | H-Trp-OBzl | 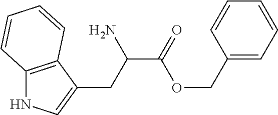 |

The employed receptor phases was polyvinyl amine-coated spherical silica gel with a particle size of 20 μm and a pore diameter of 1000 Å. In the coating method, firstly, the amino phase A was produced. The derivatized receptor phases B to K were produced from the amino phase A by means of solid phase synthesis according to known methods. The phases are summarized in Table 12:

TABLE 12
Structure of the employed receptor phases

| phase name | phase composition | phase structure |
|---|---|---|
| A BV 03002 | K1000-PVA-FA-2-5-Dod amino phase | 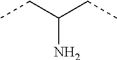 |
| B ND 03001#2 | K1000-PVA-FA-2-5-Dod-Ac-100 acetyl phase | 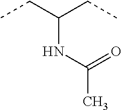 |
| C ND 03105 | K1000-PVA-FA-2-10-Dod-MVS-100 4-methylvaleryl phase | 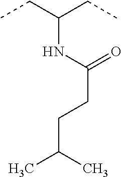 |
| D ND 03017#3 | K1000-PVA-FA-2-5-Dod-BzlO-100 benzyloxycarbonyl phase | 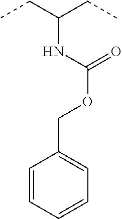 |
| I ND 02061#2 | K1000-PVA-FA-2-5-Dod-BSr-100 succinic acid phase | 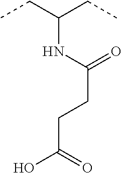 |
| J ND 03096 | K1000-PVA-FA-2-5-Dod-MVS -50- BSr-50 phase with 4-methylvaleryl groups and succinic acid groups | 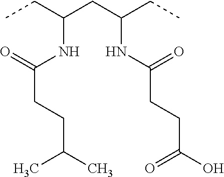 |
| K ND 03088 | K1000-PVA-FA-2-5-Dod-BzlO-50- BSr-50 phase with benzyloxycarbonyl groups and succinic acid groups | 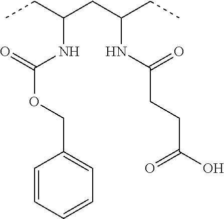 |

As mobile phase for the chromatographical tests, aqueous 10 mM tris-HCl-buffer having pH 7.5 was used.

As measure for the strength of the interaction between substrate and receptor in the respective buffer solutions, the device-independent relative elution factor k′ (capacity factor) was used. It can be calculated from the difference of elution volume at the peak maximum and the column dead volume divided by the column dead volume:

$${k}^{′} = \frac{elution volume - column dead volume}{column dead volume}$$

The k′-values of the substrates in 10 mmolar tris-HCl buffer are summarized in Table 13:

TABLE 13
k′-values of the substrates in 10 mmolar tris-HCl-buffer

| | | k′-values based on receptor phase | | | | | | |
|---|---|---|---|---|---|---|---|---|
| substrate | | A | B | C | D | I | J | K |
| 1 | H-Ala-OMe | 0.0 | 0.0 | 0.0 | 0.2 | 13.7 | 8.7 | 11.6 |
| 2 | H-Ala-OBzl | 0.0 | 0.0 | 0.2 | 2.1 | 17.4 | 13.4 | 23.9 |
| 3 | H-Leu-OMe | 0.0 | 0.0 | 0.0 | 0.3 | 12.5 | 7.1 | 10.4 |
| 4 | H-Leu-OBzl | 0.0 | 0.1 | 6.3 | 10.0 | 13.1 | 18.6 | 41.7 |
| 5 | H-Pro-OMe | 0.0 | 0.0 | 0.0 | 0.4 | — | 11.9 | 15.9 |
| 6 | H-Pro-OBzl | 0.0 | 0.1 | 0.2 | 3.6 | 21.7 | 16.6 | 34.4 |
| 7 | Z-Lys-OMe | 0.0 | 0.1 | 0.0 | 2.4 | 19.7 | 20.9 | 61.5 |
| 8 | H-Lys(Z)-OMe | 0.0 | 0.1 | 0.6 | 6.5 | 12.0 | 11.5 | 30.2 |
| 9 | Boc-Lys-OMe | 0.0 | 0.0 | 0.0 | 0.4 | 14.3 | 11.9 | 20.2 |
| 10 | H-Lys(Boc)-OMe | 0.0 | 0.0 | 0.1 | 0.5 | 9.0 | 5.3 | 9.8 |
| 11 | H-His-OMe | 0.0 | 0.0 | 0.0 | 0.3 | 18.2 | 5.7 | 13.8 |
| 12 | Bzl-His-OMe | 0.0 | 0.0 | 0.5 | 1.2 | 4.5 | 1.9 | 3.7 |
| 13 | H-Phe-OMe | 0.0 | 0.0 | 0.1 | 0.2 | 6.5 | 1.7 | 3.2 |
| 14 | H-Phe-OBzl | 0.1 | 0.1 | 12.6 | 39.0 | 7.3 | 12.3 | 39.4 |
| 15 | H-Tyr-OMe | 0.0 | 0.2 | 0.7 | 1.0 | 8.7 | 4.8 | 6.5 |
| 16 | H-Tyr-OBzl | 0.1 | 0.3 | 16.7 | 20.3 | 9.4 | 16.3 | 16.5 |
| 17 | H-Trp-OMe | 0.5 | 0.2 | 1.0 | 4.4 | 12.5 | 10.7 | 17.7 |
| 18 | H-Trp-OBzl | 0.8 | 0.3 | 49.6 | 55.4 | 16.6 | 49.5 | 186.4 |

1. Observation: In Example 1, the k′-values of amino acid derivatives with carboxylate groups were tested on amino phases. The monocarboxylates Ac-Gln 1 and Boc-Gln 2 from Example 1 achieved k′-factors of 9.5 and 8.8 on an amino phase (BV 02042). In present Example 6, one obtained for simple monoamines such as H-ala-OMe 1 and H-leu-OMe 3 k′-values of 13.7 and 12.5 on the carboxylate phase I.

Interpretation of the observation: In interchanging the interaction groups in substrate and receptor phase, the k′-values changed only little. This could be expected because the strength of the bond should be independent on the direction of the bond. For the planned application of interaction groups, it is important that a comparable binding takes place, independently which group is fixed in the recepfor or is mobile in the substrate.

2. Observation: On the amino phase A and the acetamido phase B, virtually no retention of the substrates took place.

Interpretation of the observation: The receptor phases A and B do not contain receptor groups with which a noteworthy interaction to the substrates would be possible in the selected buffer. Accordingly, the k′-values were approximately zero. These phases can be used as zero-points on a relative interaction scale. The lipophilic influence of the polymer scaffold can be neglected in the binding balance.

3. Observation: All substrates indicated a clear retention on the carboxylate phase I. The k′-values were between 4.5 to 21.7.

Interpretation of the observation: All tested substrates contain at least one amino group. Said amino group is largely protonated at pH 7.5 and can undergo strongly ionic interactions with the carboxylate anions of the phase.

4. Observation: The receptor phases C and D indicated lower retention with substrates containing a single lipophilic partial structure, for example 2, 6, 7, 8, 15, or 17. Strong retention (k′-values>8) were found with substrates which at least possessed two bigger lipophilic molecule portions, such as 4, 14, 16, and 18. Thereby, the binding to the aromatic receptor phase D was in each case higher than to the alkyl receptor phase.

Interpretation of the observation: The receptor phases C and D can only undergo lipophilic interactions. These bonds are relatively weak compared to ionic interactions. Monovalent lipophilic interactions are often at the limit of detection in the selected buffer. Substrates with two extended lipophilic residues show an increased retention as a consequence of the lipophilic contact region.

5. Observation: In most cases, the highest k′-value for the respective substrate was found on the receptor phase K.

Interpretation of the observation: The receptor phase K contains in approximately equal molar amounts carboxylate groups and benzyloxylcarbonyl groups, i.e. receptor groups for ionic and for lipophilic interactions. Since the total number of the interaction groups approximately corresponds to that one of the genuine receptor phases C, D or I, one should expect a k′-value between the k′-values of the phases D and I on the mixed receptor phase K. The detected high k′-values on the mixed receptor phase indicate that in said cases ionic and lipophilic bindings simultaneously take place, and therewith a mixed, bivalent binding mode is present.

For the strength of π-π contacts between aromatic systems, the binding of all substrates having an aromatic residue, to the benzyl group-containing phase, is stronger than to the phase J having a branched alkyl residue.

Result: With the above described experiments, it could be clearly evidenced that one could targetedly activate and deactivate interactions between a substrate and a receptor phase by suited choice of genuine receptor groups. For the regulation of affinity and selectivity, additionally the solvent composition, the ion strength and the pH can be varied.

If a substrate has two lipophilic molecule portions, it can be bivalently interact with the receptor phase what leads to a significant strengthening of the bond. In such case, it is a bivalent interaction of the same type.

It could also be shown that bivalent interactions of different type are possible (ionic and lipophilic), if both the receptor phase and the substrate contain corresponding complementary groups. Here, also a selectively binding strengthening takes place.

By design of a receptor being accordingly complementary to a target substance, accompanying substances or by-products can be easily separated off. The measure for the feasibility of the separation is the quotient from the k′-values, the selectivity alpha:

Selectivity: alpha=k₂′/k₁′

For example, with the benzyl/receptor phase D, a chromatographical separation of Boc-lys-OMe (9) and H-lys(Boc)-OMe (10) would hardly be possible. On the carboxylate receptor phase I, an alpha value of 1.59 resulted. The mixed receptor phases J and K already indicated alpha-values of 2.25 and 2.06. This was standing for the significant improvement of the chromatographical separability of a mixture by suited design of the receptor phase.

1. A method for the manufacture of at least one sorbent having at least two different groups, which are capable of binding, for the selective binding of a substrate, characterized in that it comprises the steps (i) to (ii):
(i) determining at least two groups capable of binding a sorbent from a synthetic or natural first substrate,
(ii) respectively applying at least two different groups capable of binding a to second synthetic or natural substrate to one respective carrier, thereby forming at least one sorbent, whereby the groups are the same groups of step (i) or are groups that are complementary thereto, and the second substrate of step (ii) is the same or different from the first substrate according to step (i),
and whereby the groups are determined such that the contributions of the Gibbs energies of the individual groups to the non-covalent bond with the second substrate yield a negative value of the Gibbs energy ΔG, such that a binding strengthening occurs that results in an improved separation selectivity with respect to at least one substance to be separated off.

2. A method for the selectively binding of a substrate having at least two different groups, which are capable of binding, to at least one sorbent, characterized in that it comprises the steps (i) to (iv):
(i) determining at least two groups capable of binding a sorbent from a synthetic or natural first substrate,
(ii) respectively applying at least two different groups capable of binding a second synthetic or natural substrate to one respective carrier, thereby forming at least one sorbent, whereby the groups are the same groups of step (i) or are groups that are complementary thereto, and the second substrate of step (ii) is the same or different from the first substrate according to step (i),
(iii) contacting the at least one second substrate that is the same or different from the first substrate according to (i) with at least one sorbent of step (ii),
(iv) testing the binding strength of the at least one second substrate to the at least one sorbent of step (iii),
and whereby the groups are determined such that the contributions of the Gibbs energies of the individual groups to the non-covalent bond with the second substrate yield a negative value of the Gibbs energy ΔG, such that a binding strengthening occurs that results in an improved separation selectivity with respect to at least one substance to be separated off.

3. A method according to claim 1 or 2, characterized in that the determination in step (i) comprises the dissection of a synthetic or natural first substrate into at least two components having at least two groups capable of binding a sorbent.

4. A method according to any one of claims 1 to 3, characterized in that one component has at least two different groups capable of binding.

5. A method according to any one of the preceding claims, characterized in that the at least one first substrate is the same as the at least one second substrate, and the at least two different groups capable of binding the second substrate, respectively, are selected among those groups that are complementary to the groups which are determined in step (i).

6. A method according to any one of claims 1 to 4, characterized in that the at least one first substrate is different from the at least one second substrate, and the at least two different groups capable of binding the second substrate, respectively, are selected among those groups that are complementary to the groups which are determined in step (i).

7. A method according to any one of claims 1 to 4, characterized in that the at least two groups capable of binding the at least one second substrate are selected among the groups that are determined according to step (i).

8. A method according to any one of the preceding claims, characterized in that the selection of at least two groups capable of binding a sorbent from a synthetic or a natural first substrate in step (i) yields two components each having at least one group capable of binding the sorbent, and in step (ii) one sorbent is obtained; or the selection of at least two groups capable of binding a sorbent from a synthetic or natural first substrate in step (i) yields three components each having at least one group capable of binding the sorbent, and in step (ii) at least three sorbents are obtained; or the selection of at least two groups capable of binding a sorbent from a synthetic or natural first substrate in step (i) yields four components each having at least one group capable of binding the sorbent, and in step (ii) at least six sorbents are obtained.

9. A method according to any one of the preceding claims, characterized in that the at least two different groups capable of binding of the at least one sorbent are selected among groups which are part of amino acids, sugars, nucleotides, nucleosides, pyrimidine bases and purine bases.

10. A method according to any one of the preceding claims, characterized in that the at least two different groups capable of binding of the at least one second substrate are selected among groups which are part of amino acids, sugars, nucleotides, nucleosides, pyrimidine bases and purine bases.

11. A method according to any one of the preceding claims, characterized in that the at least two different groups in step (ii), respectively, are covalently bonded to a polymer.

12. A method according to claim 11, characterized in that the polymer is directly synthesized on the carrier by means of polymerization or polycondensation of at least one monomer having at least two different groups capable of binding, or of at least two monomers each having at least one group capable of binding, whereby said groups are different.

13. A method according to any one of claims 1 to 10, characterized in that in step (ii) the at least two different groups capable of binding a second substrate are applied onto a carrier via a reagent which is selected from the group comprising activating reagents, silanization reagents and spacer, or mixtures of two or more of said reagents.

14. A method according to claim 13, characterized in that in step (ii) the at least two different groups capable of binding a second substrate are selected from the group consisting of phenyl, hydroxyphenyl, carboxyl, amine, amide, hydroxyl, indole, imidazole and guanidine residue.

15. A method according to any one of the preceding claims, characterized in that it additionally comprises the step (v):
(v) isolating the at least one second substrate.

16. A method according to any one of the preceding claims, characterized in that it additionally comprises the step (vi):
(vi) characterizing and identifying the at least one second substrate.

17. A method according to any one of the preceding claims, characterized in that the substrate comprises one or more natural agents selected from the group comprising amino acids, oligopeptides, nucleotides, proteins, glycoproteins, antigens, antibodies, carbohydrates, enzymes, co-enzymes, hormones, alkaloids, steroids, viruses, microorganisms, substances contained in vegetable and animal tissue, cells, cell fragments, cell compartments, cell disruptions, lectins, flavylium compounds, flavones, and isoflavones, or one or more synthetic agents selected from the group of substances having influence on the nervous system, having influence on the hormone system, having influence on mediators, having influence on the cardio-vascular system, having influence on the respiratory tract, having influence on the gastrointestinal tract, having influence on the kidney and the lower urinary tract, having influence on the eye, having influence on the skin, substances for the prophylaxis and therapy of infection diseases, having influence on malignant tumors, having influence on the immune system and substances having immunological influence, as well as insecticides, herbicides, pesticides and fungicides.

18. A combinatorial library comprising a collection of sorbents having at least two different first groups, respectively, that are capable of non-covalently binding at least one substrate having at least two different second groups, whereby the at least two different first groups are selected such that the contributions of the Gibbs energies of the individual first groups to the non-covalent bond with the at least two different second groups yield a total of a negative value of the Gibbs energy ΔG, such that a binding strengthening occurs which results in an improved separation selectivity with respect to at least one substance to be separated off.

19. A combinatorial library according to claim 18, characterized in that the at least two different groups of the sorbents and the at least two different groups of the at least one substrate are selected among groups which are part of amino acids, sugars, nucleotides, nucleosides, pyrimidine bases and purine bases.

20. A sorbent/substrate complex comprising at least one sorbent and at least one substrate, having at least two different second groups, whereby said sorbent has at least two different first groups capable of non-covalently binding the at least one substrate having the at least two different second groups, whereby the at least two first groups are selected such that the contributions of the Gibbs energies of the individual first groups to the non-covalent bond with the at least two different second-groups yield a total of a negative value of the Gibbs energy ΔG, such that a binding strengthening occurs which results in an improved separation selectivity with respect to at least one substance to be separated off.

21. A use of a method according to any one of claims 2 to 17, or a use of a combinatorial library according to any one of claims 18 to 20 for the detection of receptor/agent interactions, for the screening of agents, for the development of lead substances, for separating off substrates, for the purification of substrates, for the separation of isomeric compounds, for the purification of liquids by separating off harmfull substances, for the depletion of dynamically combinatorial libraries.
